SEQUENCE LISTING (14 sequences)

SEQ ID NO: 1 - protein, 15 aa, Artificial
  Synthetic Peptide
  Xaa (3)..(3): Xaa is 1Nal
  Xaa (7)..(7): Xaa is HArg
  Xaa (13)..(13): Xaa is HyP
CPXDCMXDWSTPXWC

SEQ ID NO: 2 - protein, 15 aa, Artificial
  Synthetic Peptide
  Xaa (2)..(2): Xaa is HyP
  Xaa (14)..(14): Xaa is HArg
CXLVNPLCLHPDWXC

SEQ ID NO: 3 - protein, 15 aa, Artificial
  Synthetic Peptide
CSAGWLTMCQKLHLC

SEQ ID NO: 4 - protein, 13 aa, Artificial
  Synthetic Peptide
  Xaa (3)..(3): Xaa is HArg
  Xaa (7)..(7): Xaa is tBuAla
  Xaa (11)..(11): Xaa is HArg
CVXECAXLFPXTC

SEQ ID NO: 5 - protein, 13 aa, Artificial
  Synthetic Peptide
  Xaa (3)..(3): Xaa is Cba
CYXPDYLCADEYC

SEQ ID NO: 6 - protein, 13 aa, Artificial
  Synthetic Peptide
CYLPDYLCGDEYC

SEQ ID NO: 7 - protein, 13 aa, Artificial
  Synthetic Peptide
CDLTTHNCQWGIC

SEQ ID NO: 8 - protein, 14 aa, Artificial
  Synthetic Peptide
CNLQAPCMQTGKVC

SEQ ID NO: 9 - protein, 12 aa, Artificial
  Synthetic Peptide
CNLQNPCMKFPC

SEQ ID NO: 10 - protein, 13 aa, Artificial
  Synthetic Peptide
  Xaa (9)..(9): Xaa is dK(PYA)
CYLPDYLCXDEYC

SEQ ID NO: 11 - protein, 15 aa, Artificial
  Synthetic Peptide
CLLHDHCPNTHPKLC

SEQ ID NO: 12 - protein, 14 aa, Artificial
  Synthetic Peptide
CVGLEELGPCSDLC

SEQ ID NO: 13 - protein, 13 aa, Artificial
  Synthetic Peptide
CRWHFSEPCGAWC

SEQ ID NO: 14 - protein, 13 aa, Artificial
  Synthetic Peptide
CRWSVEDPCGAWC

(19) United States
(12) Patent Application Publication
(10) Pub. No.: US 2024/0197897 A1
(43) Pub. Date: Jun. 20, 2024

(54) HETEROTANDEM BICYCLIC PEPTIDE COMPLEXES

(71) Applicant: Bicycle Tx Limited, Cambridge (GB)

(72) Inventors: Nicholas KEEN, Carlisle, MA (US); Gemma MUDD, Cambridge (GB); Phil BRANDISH, Needham, MA (US); Katie GAYNOR, Cambridge (GB); Liuhong CHEN, Cambridge (GB); Fay DUFORT, Cambridge (GB); Chris LEITHEISER, Cambridge (GB); Kevin MCDONNELL, Lexington, MA (US); Liz REPASH, Cambridge (GB); Sandra UHLENBROICH, Cambridge (GB)

(21) Appl. No.: 18/271,360

(22) PCT Filed: Jan. 10, 2022
(86) PCT No.: PCT/GB2022/050044

§ 371 Date: Jul. 7, 2023

Related U.S. Application Data
(60) Provisional application No. 63/135,273, filed on Jan. 8, 2021.
(60) Provisional application No. 63/262,599, filed on Oct. 15, 2021.

(51) Int. Cl. A61K 47/66; A61P 35/00

(52) U.S. Cl. CPC A61K 47/66; A61P 35/00

(57) ABSTRACT

The present invention relates to heterotandem bicyclic peptide complexes which comprise a first peptide ligand, which binds to a component present on a cancer cell, conjugated via a linker to one or more second peptide ligands, which bind to one or more components present on a natural killer (NK) cell. The invention also relates to the use of said heterotandem bicyclic peptide complexes in preventing, suppressing or treating cancer.

### FIELD OF THE INVENTION

The present invention relates to heterotandem bicyclic peptide complexes which comprise a first peptide ligand, which binds to a component present on a cancer cell, conjugated via a linker to one or more second peptide ligands, which bind to one or more components present on a natural killer (NK) cell. The invention also relates to the use of said heterotandem bicyclic peptide complexes in preventing, suppressing or treating cancer.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51).

The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

According to a first aspect of the invention, there is provided a heterotandem bicyclic peptide complex comprising:
(a) a first peptide ligand which binds to a component present on a cancer cell; conjugated via a linker to
(b) one or more second peptide ligands which bind to one or more components present on a natural killer (NK) cell;

wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

According to a further aspect of the invention, there is provided a pharmaceutical composition comprising a heterotandem bicyclic peptide complex as defined herein in combination with one or more pharmaceutically acceptable excipients.

According to a further aspect of the invention, there is provided a heterotandem bicyclic peptide complex as defined herein for use in preventing, suppressing or treating cancer.

### BRIEF DESCRIPTION OF THE FIGURES

FIG. 1: Activation of NK cells by BCY15664 and BCY15911 as measured by the upregulation of CD107a.

FIGS. 2 to 4: NK Cytotoxicity Assay Results for BCY15664, BCY15923 and BCY17226.

FIG. 5: IFNγ Secretion Assay Results for BCY17226.

FIGS. 6 to 10: NK Cytotoxicity Assay Results for BCY17225, BCY21686, BCY21687, BCY17231, BCY17235, BCY18731, BCY20793, BCY15924, BCY18042, BCY18049, BCY 18603, and BCY18604.

FIGS. 11 to 12: Cytokine Secretion Assay Results for BCY17225, BCY21686, BCY21687, and BCY18048.

### DETAILED DESCRIPTION OF THE INVENTION

According to a first aspect of the invention, there is provided a heterotandem bicyclic peptide complex comprising:
(a) a first peptide ligand which binds to a component present on a cancer cell; conjugated via a linker to
(b) one or more second peptide ligands which bind to one or more components present on a natural killer (NK) cell;

wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

### First Peptide Ligands

References herein to the term “cancer cell” includes any cell which is known to be involved in cancer. Cancer cells are created when the genes responsible for regulating cell division are damaged. Carcinogenesis is caused by mutation and epimutation of the genetic material of normal cells, which upsets the normal balance between proliferation and cell death. This results in uncontrolled cell division and the evolution of those cells by natural selection in the body. The uncontrolled and often rapid proliferation of cells can lead to benign or malignant tumors (cancer). Benign tumors do not spread to other parts of the body or invade other tissues. Malignant tumors can invade other organs, spread to distant locations (metastasis) and become life-threatening.

In one embodiment, the cancer cell is selected from an HT1080, A549, SC-OV-3, PC3, HT1376, NCI-H292, LnCap, MC38, MC38 #13, 4T1-D02, H322, HT29, T47D and RKO tumor cell.

In one embodiment, the component present on a cancer cell is Nectin-4.

Nectin-4 is a surface molecule that belongs to the nectin family of proteins, which comprises 4 members. Nectins are cell adhesion molecules that play a key role in various biological processes such as polarity, proliferation, differentiation and migration, for epithelial, endothelial, immune and neuronal cells, during development and adult life. They are involved in several pathological processes in humans. They are the main receptors for poliovirus, herpes simplex virus and measles virus. Mutations in the genes encoding Nectin-1 (PVRL1) or Nectin-4 (PVRL4) cause ectodermal dysplasia syndromes associated with other abnormalities. Nectin-4 is expressed during foetal development. In adult tissues its expression is more restricted than that of other members of the family. Nectin-4 is a tumor-associated antigen in 50%, 49% and 86% of breast, ovarian and lung carcinomas, respectively, mostly on tumors of bad prognosis. Its expression is not detected in the corresponding normal tissues. In breast tumors, Nectin-4 is expressed mainly in triple-negative and ERBB2+ carcinomas. In the serum of patients with these cancers, the detection of soluble forms of Nectin-4 is associated with a poor prognosis. Levels of serum Nectin-4 increase during metastatic progression and decrease after treatment. These results suggest that Nectin-4 could be a reliable target for the treatment of cancer. Accordingly, several anti-Nectin-4 antibodies have been described in the prior art. In particular, Enfortumab Vedotin (ASG-22ME) is an antibody-drug conjugate (ADC) targeting Nectin-4 and is currently clinically investigated for the treatment of patients suffering from solid tumors.

In one embodiment, the first peptide ligand comprises a Nectin-4 binding bicyclic peptide ligand.

Suitable examples of Nectin-4 binding bicyclic peptide ligands are disclosed in WO 2019/243832, the peptides of which are incorporated herein by reference.

In one embodiment, the Nectin-4 binding bicyclic peptide ligand comprises an amino acid sequence which is:
CᵢP[1Nal][dD]CᵢᵢM[HArg]DWSTP[HyP]WCᵢᵢᵢ (SEQ ID NO: 1; herein referred to as BCY8116).

wherein Cᵢ, Cᵢᵢ and Cᵢᵢᵢ represent first, second and third cysteine residues, respectively, 1Nal represents 1-naphthylalanine, HArg represents homoarginine and HyP represents trans-4-hydroxy-L-proline, or a pharmaceutically acceptable salt thereof.

In an alternative embodiment, the component present on a cancer cell is EphA2.

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) control cell positioning and tissue organization (Poliakov et al. (2004) Dev Cell 7, 465-80). Functional and biochemical Eph responses occur at higher ligand oligomerization states (Stein et al. (1998) Genes Dev 12, 667-678).

Among other patterning functions, various Ephs and ephrins have been shown to play a role in vascular development. Knockout of EphB4 and ephrin-B2 results in a lack of the ability to remodel capillary beds into blood vessels (Poliakov et al., supra) and embryonic lethality. Persistent expression of some Eph receptors and ephrins has also been observed in newly-formed, adult micro-vessels (Brantley-Sieders et al. (2004) Curr Pharm Des 10, 3431-42; Adams (2003) J Anat 202, 105-12).

The de-regulated re-emergence of some ephrins and their receptors in adults also has been observed to contribute to tumor invasion, metastasis and neo-angiogenesis (Nakamoto et al. (2002) Microsc Res Tech 59, 58-67; Brantley-Sieders et al., supra). Furthermore, some Eph family members have been found to be over-expressed on tumor cells from a variety of human tumors (Brantley-Sieders et al., supra); Marme (2002) Ann Hematol 81 Suppl 2, S66; Booth et al. (2002) Nat Med 8, 1360-1).

EPH receptor A2 (ephrin type-A receptor 2) is a protein that in humans is encoded by the EPHA2 gene.

EphA2 is upregulated in multiple cancers in man, often correlating with disease progression, metastasis and poor prognosis e.g.: breast (Zelinski et al (2001) Cancer Res. 61, 2301-2306; Zhuang et al (2010) Cancer Res. 70, 299-308; Brantley-Sieders et al (2011) PLOS One 6, e24426), lung (Brannan et al (2009) Cancer Prev Res (Phila) 2, 1039-1049; Kinch et al (2003) Clin Cancer Res. 9, 613-618; Guo et al (2013) J Thorac Oncol. 8, 301-308), gastric (Nakamura et al (2005) Cancer Sci. 96, 42-47; Yuan et al (2009) Dig Dis Sci 54, 2410-2417), pancreatic (Mudali et al (2006) Clin Exp Metastasis 23, 357-365), prostate (Walker-Daniels et al (1999) Prostate 41, 275-280), liver (Yang et al (2009) Hepatol Res. 39, 1169-1177) and glioblastoma (Wykosky et al (2005) Mol Cancer Res. 3, 541-551; Li et al (2010) Tumor Biol. 31, 477-488).

The full role of EphA2 in cancer progression is still not defined although there is evidence for interaction at numerous stages of cancer progression including tumor cell growth, survival, invasion and angiogenesis. Downregulation of EphA2 expression suppresses tumor cancer cell propagation (Binda et al (2012) Cancer Cell 22, 765-780), whilst EphA2 blockade inhibits VEGF induced cell migration (Hess et al (2001) Cancer Res. 61, 3250-3255), sprouting and angiogenesis (Cheng et al (2002) Mol Cancer Res. 1, 2-11; Lin et al (2007) Cancer 109, 332-40) and metastatic progression (Brantley-Sieders et al (2005) FASEB J. 19, 1884-1886).

An antibody drug conjugate to EphA2 has been shown to significantly diminish tumor growth in rat and mouse xenograft models (Jackson et al (2008) Cancer Research 68, 9367-9374) and a similar approach has been tried in man although treatment had to be discontinued for treatment related adverse events (Annunziata et al (2013) Invest New drugs 31, 77-84).

In one embodiment, the first peptide ligand comprises an EphA2 binding bicyclic peptide ligand.

Suitable examples of EphA2 binding bicyclic peptide ligands are disclosed in WO 2019/122860, WO 2019/122861 and WO 2019/122863, the peptides of which are incorporated herein by reference.

In one embodiment, the EphA2 binding bicyclic peptide ligand comprises an amino acid sequence which is:

(SEQ ID NO: 2)
Cᵢ[HyP]LVNPLCᵢᵢLHP[dD]W[HArg]Cᵢᵢᵢ;

wherein Cᵢ, Cᵢᵢ and Cᵢᵢᵢ represent first (i), second (ii) and third (iii) reactive groups HyP represents trans-4-hydroxy-L-proline, HArg represents homoarginine, or a pharmaceutically acceptable salt thereof.

In a further embodiment, the EphA2 binding bicyclic peptide ligand optionally comprises N-terminal and/or C-terminal modifications and comprises:
A-[HArg]-D-(SEQ ID NO: 2) (herein referred to as BCY9594);

wherein HArg represents homoarginine, or a pharmaceutically acceptable salt thereof.

In an alternative embodiment, the component present on a cancer cell is PD-L1.

Programmed cell death 1 ligand 1 (PD-L1) is a 290 amino acid type I transmembrane protein encoded by the CD274 gene on mouse chromosome 19 and human chromosome 9. PD-L1 expression is involved in evasion of immune responses involved in chronic infection, e.g., chronic viral infection (including, for example, HIV, HBV, HCV and HTLV, among others), chronic bacterial infection (including, for example, Helicobacter pylori, among others), and chronic parasitic infection (including, for example, Schistosoma mansoni). PD-L1 expression has been detected in a number of tissues and cell types including T-cells, B-cells, macrophages, dendritic cells, and nonhaematopoietic cells including endothelial cells, hepatocytes, muscle cells, and placenta.

PD-L1 expression is also involved in suppression of anti-tumor immune activity. Tumors express antigens that can be recognised by host T-cells, but immunologic clearance of tumors is rare. Part of this failure is due to immune suppression by the tumor microenvironment. PD-L1 expression on many tumors is a component of this suppressive milieu and acts in concert with other immunosuppressive signals. PD-L1 expression has been shown in situ on a wide variety of solid tumors including breast, lung, colon, ovarian, melanoma, bladder, liver, salivary, stomach, gliomas, thyroid, thymic epithelial, head, and neck (Brown J A et al. 2003 Immunol. 170:1257-66; Dong H et al. 2002 Nat. Med. 8:793-800; Hamanishi J, et al. 2007 Proc. Natl. Acad. Sci. USA 104:3360-65; Strome S E et al. 2003 Cancer Res. 63:6501-5; Inman B A et al. 2007 Cancer 109:1499-505; Konishi J et al. 2004 Clin. Cancer Res. 10:5094-100; Nakanishi J et al. 2007 Cancer Immunol. Immunother. 56:1173-82; Nomi T et al. 2007 Clin. Cancer Res. 13:2151-57; Thompson R H et al. 2004 Proc. Natl. Acad. Sci. USA 101: 17174-79; Wu C et al. 2006 Acta Histochem. 108:19-24). In addition, the expression of the receptor for PD-L1, Programmed cell death protein 1 (also known as PD-1 and CD279) is upregulated on tumor infiltrating lymphocytes, and this also contributes to tumor immunosuppression (Blank C et al. 2003 Immunol. 171:4574-81). Most importantly, studies relating PD-L1 expression on tumors to disease outcome show that PD-L1 expression strongly correlates with unfavourable prognosis in kidney, ovarian, bladder, breast, gastric, and pancreatic cancer (Hamanishi J et al. 2007 Proc. Natl. Acad. Sci. USA 104:3360-65; Inman B A et al. 2007 Cancer 109:1499-505; Konishi J et al. 2004 Clin. Cancer Res. 10:5094-100; Nakanishi J et al. 2007 Cancer Immunol. Immunother. 56:1173-82; Nomi T et al. 2007 Clin. Cancer Res. 13:2151-57; Thompson R H et al. 2004 Proc. Natl. Acad. Sci. USA 101:17174-79; Wu C et al. 2006 Acta Histochem. 108:19-24). In addition, these studies suggest that higher levels of PD-L1 expression on tumors may facilitate advancement of tumor stage and invasion into deeper tissue structures.

The PD-1 pathway can also play a role in haematologic malignancies. PD-L1 is expressed on multiple myeloma cells but not on normal plasma cells (Liu J et al. 2007 Blood 110:296-304). PD-L1 is expressed on some primary T-cell lymphomas, particularly anaplastic large cell T lymphomas (Brown J A et al, 2003 Immunol. 170:1257-66). PD-1 is highly expressed on the T-cells of angioimmunoblastic lymphomas, and PD-L1 is expressed on the associated follicular dendritic cell network (Dorfman D M et al. 2006 Am. J. Surg. Pathol. 30:802-10). In nodular lymphocyte-predominant Hodgkin lymphoma, the T-cells associated with lymphocytic or histiocytic (L&H) cells express PD-1. Microarray analysis using a readout of genes induced by PD-1 ligation suggests that tumor-associated T-cells are responding to PD-1 signals in situ in Hodgkin lymphoma (Chemnitz J M et al. 2007 Blood 110:3226-33). PD-1 and PD-L1 are expressed on CD4 T-cells in HTLV-1-mediated adult T-cell leukaemia and lymphoma (Shimauchi T et al. 2007 Int. J. Cancer 121: 2585-90). These tumor cells are hyporesponsive to TCR signals.

Studies in animal models demonstrate that PD-L1 on tumors inhibits T-cell activation and lysis of tumor cells and in some cases leads to increased tumor-specific T-cell death (Dong H et al. 2002 Nat. Med. 8:793-800; Hirano F et al. 2005 Cancer Res. 65:1089-96). Tumor-associated APCs can also utilise the PD-1:PD-L1 pathway to control antitumor T-cell responses. PD-L1 expression on a population of tumor-associated myeloid DCs is upregulated by tumor environmental factors (Curiel T J et al. 2003 Nat. Med. 9:562-67). Plasmacytoid dendritic cells (DCs) in the tumor-draining lymph node of B16 melanoma express IDO, which strongly activates the suppressive activity of regulatory T-cells. The suppressive activity of IDO-treated regulatory T-cells required cell contact with IDO-expressing DCs (Sharma M D et al. 2007 Clin. Invest. 117:2570-82).

In one embodiment, the first peptide ligand comprises a PD-L1 binding bicyclic peptide ligand.

In one embodiment, the PD-L1 binding bicyclic peptide ligand comprises an amino acid sequence which is:

(SEQ ID NO: 3)
CᵢSAGWLTMCᵢᵢQKLHLCᵢᵢᵢ;

wherein Cᵢ, Cᵢᵢ and Cᵢᵢᵢ represent first (i), second (ii) and third (iii) cysteine groups, respectively, or a pharmaceutically acceptable salt thereof.

In a further embodiment, the molecular scaffold is TATA and the PD-L1 binding bicyclic peptide ligand optionally comprises N-terminal and/or C-terminal modifications and comprises:
Ac-D-[Harg]-(SEQ ID NO: 3)-PSH (herein referred to as BCY11865);

wherein Harg represents homoarginine, or a pharmaceutically acceptable salt thereof.

Suitable examples of PD-L1 binding bicyclic peptide ligands are disclosed in WO 2020/128526 and WO 2020/128527, the peptides of which are incorporated herein by reference.

In an alternative embodiment, the component present on a cancer cell is membrane type 1 matrix metallopeptidase 14 (MT1, also known as MMP14). MT1-MMP is a transmembrane metalloprotease that plays a major role in the extracellular matrix remodeling, directly by degrading several of its components and indirectly by activating pro-MMP2. MT1-MMP is crucial for tumor angiogenesis (Sounni et al (2002) FASEB J. 16(6), 555-564) and is over-expressed on a variety of solid tumours, therefore the MT1-MMP-binding bicycle peptides of the present invention have particular utility in the targeted treatment of cancer, in particular solid tumours such as non-small cell lung carcinomas. In one embodiment, the bicyclic peptide of the invention is specific for human MT1-MMP. In a further embodiment, the bicyclic peptide of the invention is specific for mouse MT1-MMP. In a yet further embodiment, the bicyclic peptide of the invention is specific for human and mouse MT1-MMP. In a yet further embodiment, the bicyclic peptide of the invention is specific for human, mouse and dog MT1-MMP.

In one embodiment, the MT1 binding bicyclic peptide ligand comprises an amino acid sequence which is:

(SEQ ID NO: 4)
CᵢV[Harg]ECᵢᵢA[tBuAla]LFP[Harg]TCᵢᵢᵢ;

wherein Cᵢ, Cᵢᵢ and Cᵢᵢᵢ represent first (i), second (ii) and third (iii) cysteine groups, respectively, or a pharmaceutically acceptable salt thereof.

In a further embodiment, the molecular scaffold is TATA and the MT1 binding bicyclic peptide ligand optionally comprises N-terminal and/or C-terminal modifications and comprises:
LPP-(SEQ ID NO: 4) (herein referred to as BCY14320);

or a pharmaceutically acceptable salt thereof.

Suitable examples of MT1 binding bicyclic peptide ligands are disclosed in WO 2016/067035, the peptides of which are incorporated herein by reference.

In an alternative embodiment, the component present on a cancer cell is prostate-specific membrane antigen (PSMA).

Prostate-specific membrane antigen (PSMA) (also known as Glutamate carboxypeptidase II (GCPII), N-acetyl-L-aspartyl-L-glutamate peptidase I (NAALADase I) and NAAG peptidase) is an enzyme that in humans is encoded by the FOLH1 (folate hydrolase 1) gene. Human GCPII contains 750 amino acids and weighs approximately 84 kDa.

Human PSMA is highly expressed in the prostate, roughly a hundred times greater than in most other tissues. In some prostate cancers, PSMA is the second-most upregulated gene product, with an 8- to 12-fold increase over levels in noncancerous prostate cells. Because of this high expression, PSMA is being developed as potential biomarker for therapy and imaging of some cancers. In human prostate cancer, the higher expressing tumors are associated with quicker time to progression and a greater percentage of patients suffering relapse.

In one embodiment, the first peptide ligand comprises a PSMA binding bicyclic peptide ligand.

Suitable examples of PSMA binding bicyclic peptide ligands are disclosed in WO 2019/243455 and WO 2020/120980, the peptides of which are incorporated herein by reference

### Second Peptide Ligands

It will be appreciated that the one or more second peptide ligands are required to bind to one or more components present on a natural killer (NK) cell. It will also be appreciated that when there is more than one second peptide ligand present, said second peptide ligands may bind to the same or differing targets within NK cells. Thus, in one embodiment, said second bicyclic peptide ligands are specific for the same target within the NK cell. In a further embodiment, the heterotandem bicyclic peptide complex comprises at least two identical second bicyclic peptide ligands. By “identical” it is meant second bicyclic peptides having the same amino acid sequence, most critically the same amino acid sequence refers to the binding portion of said second bicyclic peptide (for example, the sequence may vary in attachment position). In this embodiment, each of the second bicyclic peptides within the heterotandem bicyclic peptide complex will bind exactly the same epitope upon the same target of the NK cell—the resultant target bound complex will therefore create a homodimer (if the heterotandem bicyclic peptide complex comprises two identical second bicyclic peptides), homotrimer (if the heterotandem bicyclic peptide complex comprises three identical second bicyclic peptides) or homotetramer (if the heterotandem bicyclic peptide complex comprises four identical second bicyclic peptides), etc.

In an alternative embodiment, the heterotandem bicyclic peptide complex comprises at least two differing second bicyclic peptide ligands. By “differing” it is meant second bicyclic peptides having a different amino acid sequence. In this embodiment, the differing second bicyclic peptide ligands within the heterotandem bicyclic peptide complex will bind to different epitopes on NK cells—the resultant target bound complex will therefore create a biparatopic (if the heterotandem bicyclic peptide complex comprises two differing second bicyclic peptides), triparatopic (if the heterotandem bicyclic peptide complex comprises three differing second bicyclic peptides) or tetraparatopic (if the heterotandem bicyclic peptide complex comprises four differing second bicyclic peptides), etc.

Without being bound by theory it is believed that the resultant heterotandem bicyclic peptide complexes are able to activate receptors by hetero-crosslinking differing targets, such as differing target receptors on NK cells. Thus, in one embodiment, said second bicyclic peptide ligands are specific for different targets on NK cells. It will be appreciated that in this embodiment, the heterotandem bicyclic peptide complex comprises at least two differing second bicyclic peptide ligands (i.e. second bicyclic peptide ligands having differing amino acid sequences). In this embodiment, each of the second bicyclic peptides within the heterotandem bicyclic peptide complex will bind a differing epitope upon NK cells—the resultant target bound complex will therefore create a bispecific heterotandem bicyclic peptide complex (if the heterotandem bicyclic peptide complex comprises two differing second bicyclic peptides), trispecific multimeric binding complex (if the heterotandem bicyclic peptide complex comprises three differing second bicyclic peptides), tetraspecific heterotandem bicyclic peptide complex (if the heterotandem bicyclic peptide complex comprises four differing second bicyclic peptides), etc.

### NKp46 Binding Bicyclic Peptides

Natural killer (NK) cells are members of the innate immune system representing a small fraction of peripheral blood mononuclear cells. As frontline responders, these immune cells detect and eliminate unhealthy cells and bridge the innate immune response to the adaptive immune response. Due to their inherent properties, NK cells are an excellent candidate to enhance the therapeutic tools in immune oncology and autoimmunity.

NK cells are responsible for immune surveillance conducted through a variety of inhibitory and activating receptors. These activating and inhibitory receptors on the NK cellular surface are a complex means through which the activity of NK cells is kept in balance in healthy individuals. NK cells recognize the MHC class I molecules on the surface of healthy cells and are restrained through inhibitory receptors from eliminating these healthy cells. In times of stress, infection, or transformation, NK cells recognize the unhealthy cells through the loss of MHC class I on the cell surface and the induction of NK cell receptor ligands which bind to activating receptors. The recognition of non-self by the NK cells elicits a cytotoxic response, a release of cytokines and cytotoxic molecules for the elimination of the unhealthy cells.

NK cell activity is by a complex mechanism that involves both activating and inhibitory signals. Multiple reports have provided evidence for a central role of NK cell receptors in natural cytotoxicity and usefulness in the treatment of cancer. There is an unmet need for further understanding and enhancement of NK cell mediated recognition and killing of tumor cells. Reports suggest tumor cells utilize many mechanisms to reduce NK activity, and that NK cell presence and efficacy is associated with favorable prognosis in patients (Pasero et al. (2015) Oncotarget 6(16), 14360-14373, Stringaris et al. (2014) Haematologica 99(5), 836-847). It is through therapeutic intervention that one may harness the potential NK cells may play in mediating an immune response to combat cancer and autoimmune diseases.

In one embodiment, the one or more components present on a natural killer (NK) cell is a natural cytotoxicity receptor present on the NK cell surface. In a further embodiment, the one or more components present on a natural killer (NK) cell is a natural cytotoxicity receptor selected from NKp30, NKp44 and NKp46. In a yet further embodiment, the one or more components present on a natural killer (NK) cell is NKp46.

The natural cytotoxicity receptors (NCR) are a family of stimulatory receptors expressed on the NK cell surface that elicit NK activation and cell-mediated cytotoxicity. The NCR family consists of three members, NKp30, NKp44, and NKp46. Although the cellular ligand for NKp46 is unknown, a role for NKp46 in antitumor immunity has been shown. Viral antigen-mediated NKp46 activation of NK cells results in tumor rejection (Chinnery et al. 2012). Upon interaction with its ligand, the NKp46 receptor triggers NK cells to induce directed cytotoxicity, illustrated by the use of anti-NKp46 blocking antibodies inhibiting the ability of NK cells to lyse targets (Arnon et al. 2004). The amount of NCR expression on the NK cell surface also increases NK cytotoxicity. A strong correlation between the density of NCR expression and the ability of NK cells to kill target cells, including a wide variety of tumor cells, has been identified (Moretta et al. 2006). In AML and in cervical cancer and precursor lesions, the insufficient amount of NCR or NCR ligands rendered tumor cells resistant to NK cytotoxicity (Costello et al. 2002, Garcia-Iglesias et al. 2009). In many solid tumors, NK cells are downregulated by the tumor microenvironment, among which include the tumor shedding of NCR ligands and immune editing, which prevent NK cells' ability to recognize, infiltrate, and kill the tumor cells (Nayyar 2019, Stojanovic et al. 2011, Sordo-Bahamonde et al. 2020, Watanabe et al. 2010, Izawa et al. 2011, Koo et al. 2013, Sun et al. 2015, Hasmim et al. 2015, Han et al. 2018). Stringaris et al. (2014) reported downregulation of NKp46, upregulation of NK cell inhibitory receptor NKG2A and low cytotoxic capacity of NK cells from AML patients. Furthermore, in solid cancer such as prostate cancer, there was reported a decreased expression of several activating receptors (CD16, NKp30, NKp46, NKG2D and DNAM-1), and an increase in the inhibitory receptor CD85j (Pesaro et al. 2016). In contrast, Gautheir et al. (2019) has identified NKp46 as a good candidate for the targeting of an activating receptor on NK cells in cancer, demonstrating no statistically significant downregulation of NKp46 in the periphery in SCCHN, breast, liver, lung, kidney, and metastatic melanoma cancer patients. Additionally, in multiple solid tumors sustained NKp46 expression, associated with the downregulation of other activating receptors, such as NKG2D, NKp30, and NKp44, and low CD16 expression on tumor infiltrating lymphocytes has been reported for cancers, such as acute myeloid leukemia, breast cancer, and lung carcinoma (Fauriat et al. 2007, Mamessier et al. 2011, Platonova et al. 2011, Levi et al. 2015, Kim et al. 2010, MacFarlane et al. 2017). Therefore, NKp46 has shown to be a specific NK surface marker suitable for therapeutic application to identify and targeting NK cells to tumors.

In one embodiment, the one or more NKp46 binding bicyclic peptide ligands comprise an amino acid sequence which is selected from:

(SEQ ID NO: 5)
CᵢY[Cba]PDYLCᵢᵢ[dA]DEYCᵢᵢᵢ;
(SEQ ID NO: 6)
CᵢYLPDYLCᵢᵢGDEYCᵢᵢᵢ;
(SEQ ID NO: 7)
CᵢDLTTHNCᵢᵢQWGICᵢᵢᵢ;
(SEQ ID NO: 8)
CᵢNLQAPCᵢᵢMQTGKVCᵢᵢᵢ;
(SEQ ID NO: 9)
CᵢNLQNPCᵢᵢMKFPCᵢᵢᵢ;
(SEQ ID NO: 10)
CᵢYLPDYLCᵢᵢ[dK(PYA)]DEYCᵢᵢᵢ;
and
(SEQ ID NO: 11)
CᵢLLHDHCᵢᵢPNTHPKLCᵢᵢᵢ;

wherein Cᵢ, Cᵢᵢ and Cᵢᵢᵢ represent first, second and third cysteine residues, respectively, and wherein Cba represents β-cyclobutylalanine, dA represents D-Alanine, and PYA represents pentynoic acid, or a pharmaceutically acceptable salt thereof.

In a further embodiment, the molecular scaffold is TATA and the one or more NKp46 binding bicyclic peptide ligands optionally comprise N-terminal and/or C-terminal modifications and comprises:
Ac-(SEQ ID NO: 5)-[K(PYA)] (herein referred to as BCY17224);
A-(SEQ ID NO: 6)-A-[dK(PYA)] (herein referred to as BCY15452);
A-(SEQ ID NO: 7)-A-[K(PYA)] (herein referred to as BCY15686);
A-(SEQ ID NO: 8)-A-[K(PYA)] (herein referred to as BCY15687);
A-(SEQ ID NO: 9)-A-[K(PYA)] (herein referred to as BCY18004);
A-(SEQ ID NO: 10)-A (herein referred to as BCY17662); and
A-(SEQ ID NO: 11)-A-[K(PYA)] (herein referred to as BCY18005);

wherein PYA represents pentynoic acid or a pharmaceutically acceptable salt thereof.

### CD16a Binding Bicyclic Peptides

In one embodiment, the one or more components present on a natural killer (NK) cell is an Fc receptor present on the NK cell surface. In a further embodiment, the one or more components present on a natural killer (NK) cell is a low-affinity Fc gamma receptor (FcγR) selected from FcγRIIA, FcγRIIB, FcγRIIC, FcγRIIIA, and FcγRIIIB. In a yet further embodiment, the one or more components present on a natural killer (NK) cell is FcγRIIIA (also known as CD16a).

Fc receptors are expressed on the surface of many leukocytes. In humans, five classic low-affinity Fc gamma receptors (FcγRs) (FcγRIIA, FcγRIIB, FcγRIIC, FcγRIIIA, and FcγRIIIB) bind to the Fc portion of immunoglobulin G (lgG) and are mediators of inflammation via immune cell activation as well as inhibition (Muta et al. 1994, Ravetch et al. 2001). The FcγRIIIA (CD16a) is activated by engagement with the Fc portion of the IgG molecule and is critical for the antibody-dependent cell cytotoxicity (ADCC) process. ADCC is a mechanism in which antigen-specific antibodies direct NK cells to kill the antigen-expressing cancer cells (Arnould et al. 2006). Playing a vital role in the anti-tumour effects of IgG1 mAbs, several studies have shown that part of the anti-tumor effect of trastuzumab, a human IgG1 anti-human epidermal growth factor receptor 2 (HER-2) antibody, as well as the EGFR-antibody cetuximab in metastatic colorectal patients, is through ADCC (Zhang et al. 2007, 2020, Wu et al. 2003). Similar results can be described for rituximab, a chimeric IgG1 mAb for B-cell differentiation antigen CD20 (Manches et al. 2003, Clynes et al. 2000). The usefulness of CD16 expression in directing immune cells to promote tumour cell killing has been illustrated in overexpression studies. Through retroviral transduction of Ig-Fc, the expression of IgFc on the surface of B16 melanoma cells lead to tumor killing in vivo (Riddle et al. 2005). The role of FcγR engagement for directing NK cell to tumors has also been illustrated in studies whereby chimeric antigen receptor T cells express CD16 scFv and are directed to antibody coated tumor cells. The introduction of CD16-CarT in in vivo models to EGFR or CD20 tumor-bearing mice treated with cetuximab or rituximab enhanced immune cell targeting and ADCC killing thereby eradicating evasive tumors (Rataj et al. 2019, Caratelli et al. 2017).

The contributions of FcγR genes to autoimmune diseases have attracted substantial attention, and functional FcγR polymorphisms have been reported to play important roles in the pathogenesis of autoimmune diseases (Salmon et al. 2001, Morgan et al. 2003, Wu et al. 1997). FcγR-knockout mouse models indicate that both activating and inhibitory FcγRs influence the development of autoimmune diseases (Nabbe et al. 2003, Kleinau et al. 2000, Bolland et al. 2000). The variations in FcγR expression significantly affect lgG immune complex-mediated signal thresholds. Notably, proinflammatory and anti-inflammatory cytokines could modulate the expression levels of activating and inhibitory FcγRs that affect the threshold immune cell response to lgG immune complexes (Pricop et al. 2001, Boruchov et al. 2005). The FCGR3A and FCGR3B copy number variations have been associated with systemic lupus erythematosus (SLE) and rheumatoid arthritis (RA) in Taiwanese patients (Chen et al. 2014). A high FCGR3A copy number was demonstrated to be a risk factor for SLE. Higher frequencies of cytokine-producing FcγRIIIA-positive dendritic cells (DCs) were observed in SLE patients, particularly in those with active disease, suggesting that FcγRIIIA-mediated inflammatory cytokine production in DCs might contribute to disease pathogenesis (Henriques et al. 2012). It is thought that the higher density of activating FcγRIIIA on the surface of immune cells (NK cells, monocytes, DCs, macrophages, and subsets of T cells) could tip the delicate balance of immune responses toward intense inflammation, which may result in the development of SLE (Chen et al. 2014). A correlation has been demonstrated between a low FCGR3A copy number and low CD16A expression on NK cells, suggesting that FCGR3A copy number has physiologic implications in NK cell functions. Most importantly, FCGR3A deficiency is associated with 2 distinct autoimmune diseases (SLE and RA), suggesting that defective FcγRIIIA functions may represent a common risk factor for various autoimmune diseases. The disease associations suggest that modulation of FcγRIIIA function may be an important therapeutic target for lupus nephritis (Chen et al. 2014).

Therapeutic innovations have provided some hope as to a means to block inflammation in autoimmune disease through FcγR interactions. Previous studies support the potential of FcγR inhibition as a therapeutic strategy to inhibit immune complex-mediated events in autoimmune diseases (Clarkson et al. 1986, Flaherty et al. 2012). The IgG Fc fragment by itself has demonstrated good efficacy in animal models of RA and in humans with thrombocytopenia (ITP) or Kawasaki disease (Anthony et al. 2008, Debré M et al. 1993, Hsu et al. 1993). Multivalent Fc constructs have been shown to inhibit immune complex processes in murine models of thrombocytopenia and arthritis (Ortiz et al. 2016). Furthermore, the infusion of soluble FcγR3a and 2a can inhibit immune complex triggered inflammation in the murine lupus model (Li et al. 2014). The blockade of immune complex formation on NK cells is an avenue to explore for the decreased activation of inflammation and thus autoimmune diseases.

In one embodiment, the one or more CD16a binding bicyclic peptide ligands comprise an amino acid sequence which is selected from:

(SEQ ID NO: 12)
CᵢVGLEELGPCᵢᵢSDLCᵢᵢᵢ;
(SEQ ID NO: 13)
CᵢRWHFSEPCᵢᵢGAWCᵢᵢᵢ;
and
(SEQ ID NO: 14)
CᵢRWSVEDPCᵢᵢGAWCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the molecular scaffold is TBMT and the one or more CD16a binding bicyclic peptide ligands optionally comprise N-terminal and/or C-terminal modifications and comprises:
A-(SEQ ID NO: 12)-A-[K(PYA)] (herein referred to as BCY13886);
A-(SEQ ID NO: 13)-A-[K(PYA)] (herein referred to as BCY20361); and
A-(SEQ ID NO: 14)-A-[K(PYA)] (herein referred to as BCY13883);

wherein PYA represents pentynoic acid or a pharmaceutically acceptable salt thereof.

In one embodiment, the heterotandem bicyclic peptide complex comprises one (i.e. a single) second peptide ligand which binds to a component present on a natural killer (NK) cell. In a further embodiment, the single second peptide ligand is an NKp46 binding bicyclic peptide ligand as defined herein or a CD16a binding bicyclic peptide as defined herein.

In an alternative embodiment, the heterotandem bicyclic peptide complex comprises two second peptide ligands which bind to a component present on a natural killer (NK) cell. In a further embodiment, the two second peptide ligands are:
both NKp46 binding bicyclic peptide ligands as defined herein; or
both CD16a binding bicyclic peptides as defined herein; or
one NKp46 binding bicyclic peptide ligand as defined herein and one CD16a binding bicyclic peptide as defined herein.

In a yet further embodiment, when the heterotandem bicyclic peptide complex comprises two NKp46 binding bicyclic peptide ligands as defined herein, said peptide ligands are identical (i.e. share the same peptide sequence).

In a yet further embodiment, when the heterotandem bicyclic peptide complex comprises two CD16a binding bicyclic peptide ligands as defined herein, said peptide ligands are identical (i.e. share the same peptide sequence).

In an alternative embodiment, the heterotandem bicyclic peptide complex comprises three second peptide ligands which bind to a component present on a natural killer (NK) cell. In a further embodiment, the three second peptide ligands are each NKp46 binding bicyclic peptide ligands as defined herein. In a yet further embodiment, the three NKp46 binding bicyclic peptide ligands as defined herein are identical (i.e. share the same peptide sequence).

### Linkers

It will be appreciated that the first peptide ligand may be conjugated to the one or more second peptide ligands via any suitable linker. Typically, the design of said linker will be such that the two or more total Bicyclic peptides are presented in such a manner that they can bind unencumbered to their respective targets either alone or while simultaneously binding to both target receptors. Additionally, the linker should permit binding to both targets simultaneously while maintaining an appropriate distance between the target cells that would lead to the desired functional outcome. The properties of the linker may be modulated to increase length, rigidity or solubility to optimise the desired functional outcome. The linker may also be designed to permit the attachment of more than one Bicycle to the same target. Increasing the valency of either binding peptide may serve to increase the affinity of the heterotandem for the target cells or may help to induce oligomerisation of one or both of the target receptors.

In one embodiment, the linker is a linear linker. Without being bound by theory it is believed that the linear linker has the advantage of allowing the presence of one first peptide at one end and one second peptide at the other end.

In a further embodiment, the linear linker is selected from:

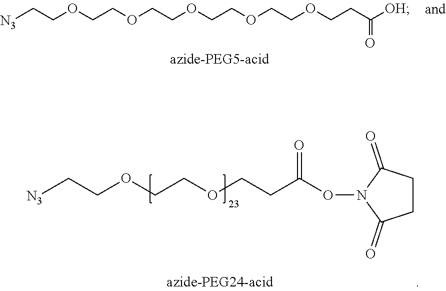

In one embodiment, the linker is a branched linker. Without being bound by theory it is believed that the branched linker has the advantage of allowing the presence of one first peptide at one end and the two or more second peptides at the other end.

In a further embodiment, the branched linker is selected from:

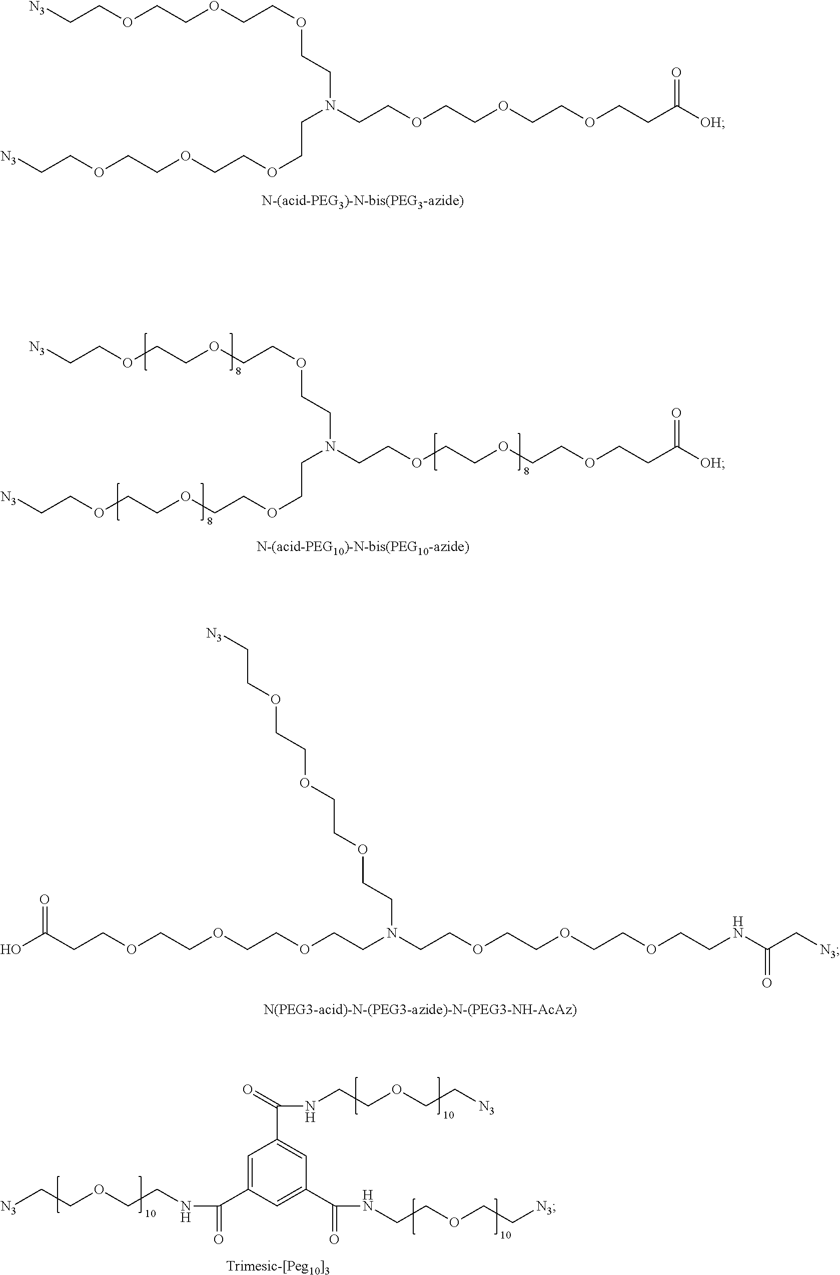

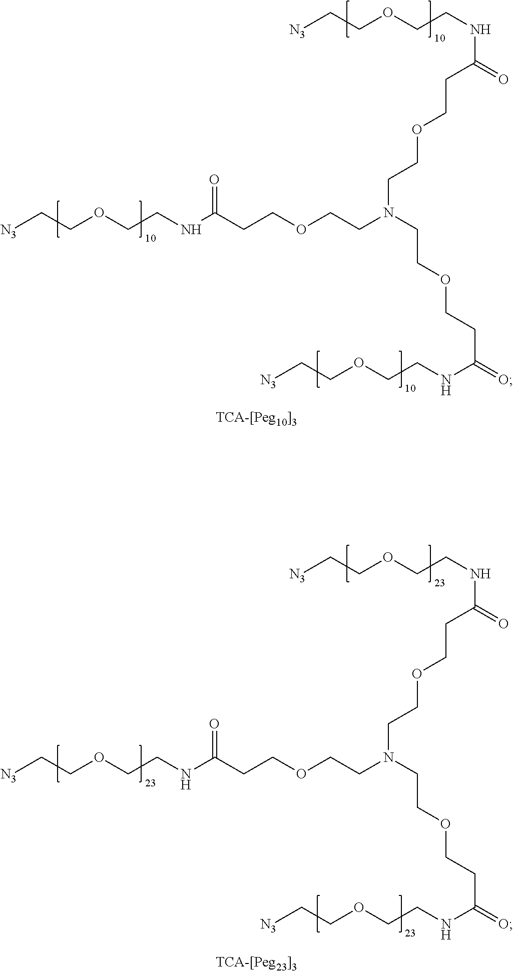

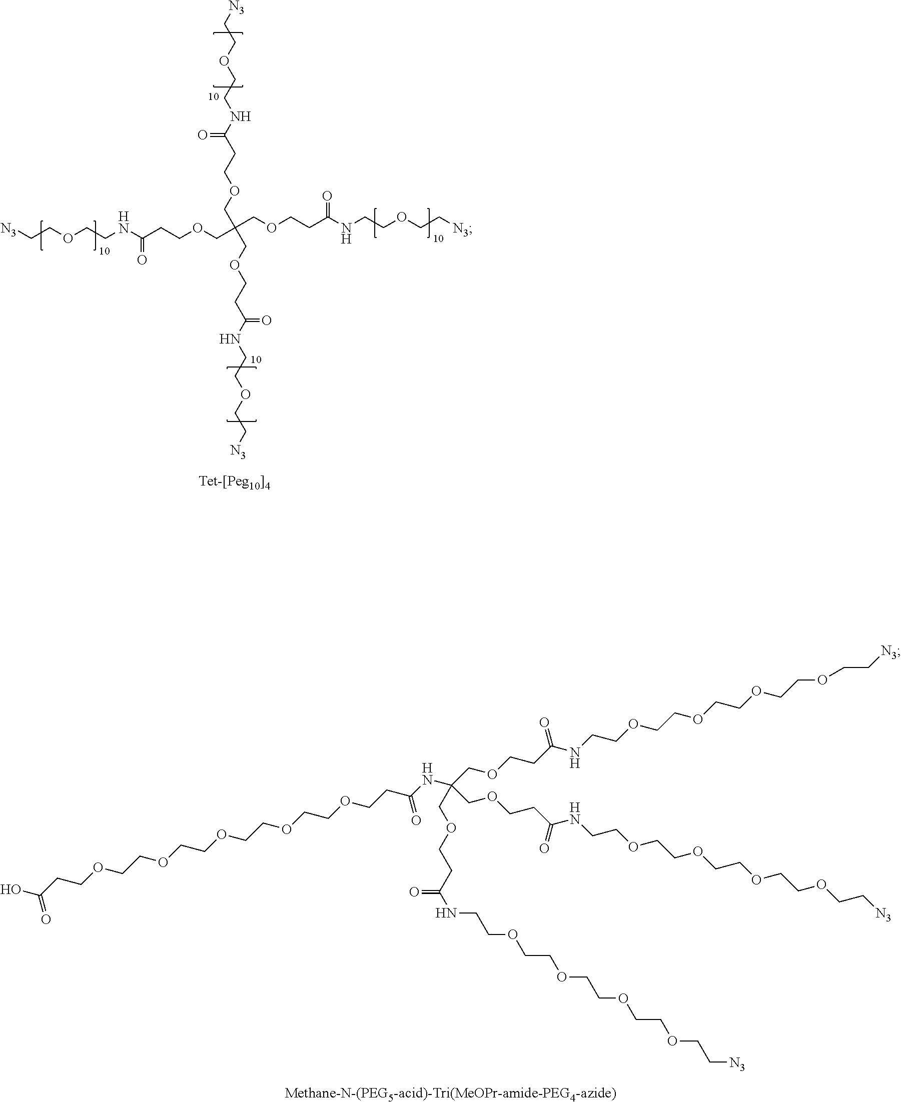

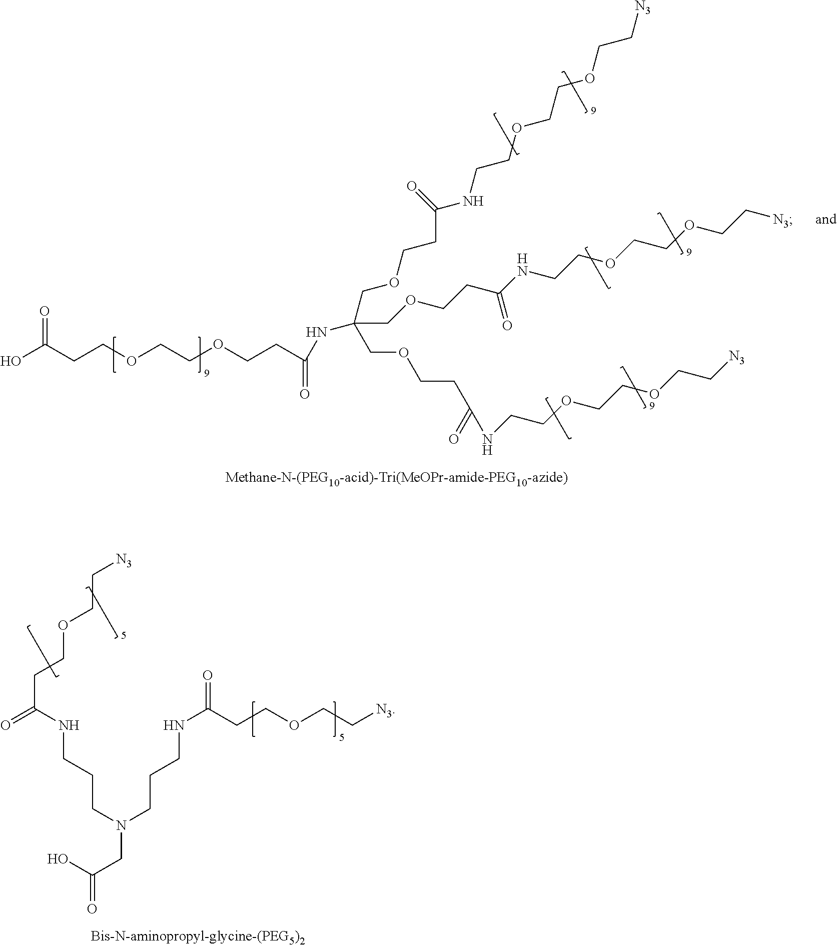

In one particular embodiment, the branched linker is:

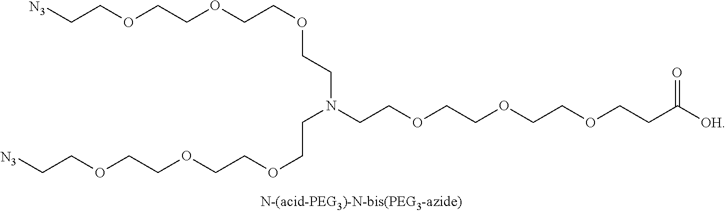 [SMILES: [N-]=[N+]=NCCOCCOCCOCCN(CCOCCOCCOCCN=[N+]=[N-])CCOCCOCCOCCC(=O)O]

### Heterotandem Complexes

In one specific embodiment, the first peptide ligand comprises an EphA2 binding bicyclic peptide ligand attached to a TATA scaffold, the one or more second peptide ligands comprise two NKp46 binding bicyclic peptide ligands attached to a TATA scaffold and said heterotandem complex is the complex listed in Table A1, Table A2 and Table A3:

TABLE A1
(EphA2:NKp46; 1:1)

| Complex | EphA2 | Attachment | | NKp46 | Attachment |
|---|---|---|---|---|---|
| No. | BCY No. | Point | Linker | BCY No. | Point |
| BCY17225 | BCY9594 | N-terminus | azide- | BCY17224 | C-terminal |
| | | | PEG5- | | Lys(PYA) |
| | | | acid | | |
| BCY18731 | BCY9594 | N-terminus | azide- | BCY17224 | C-terminal |
| | | | PEG24- | | Lys(PYA) |
| | | | acid | | |

The heterotandem bicyclic peptide complex BCY17225 consists of an EphA2 specific peptide BCY9594 linked to one NKp46 specific peptide (BCY17224) via an azide-PEG5-acid linker, shown pictorially as:

The heterotandem bicyclic peptide complex BCY18731 consists of a EphA2 specific peptide BCY9594 linked to one NKp46 specific peptide (BCY17224) via an azide-PEG24-acid linker, shown pictorially as:

TABLE A2
(EphA2:NKp46; 1:2)

| | EphA2 | Attachment | | NKp46 | Attachment |
|---|---|---|---|---|---|
| Complex No. | BCY No. | Point | Linker | BCY No. | Point |
| BCY15664 | BCY9594 | N-terminus | N-(acid-PEG₃)- | BCY15452 | C-terminal D- |
| | | | N-bis(PEG₃-azide) | | Lys(PYA) |
| BCY15923 | BCY9594 | N-terminus | N-(acid-PEG₃)- | BCY15686 | C-terminal |
| | | | N-bis(PEG₃-azide) | | Lys(PYA) |
| BCY17226 | BCY9594 | N-terminus | N-(acid-PEG₃)- | BCY17224 | C-terminal |
| | | | N-bis(PEG₃-azide) | | Lys(PYA) |
| BCY15924 | BCY9594 | N-terminus | N-(acid-PEG₃)- | BCY15687 | C-terminal |
| | | | N-bis(PEG₃-azide) | | Lys(PYA) |
| BCY18042 | BCY9594 | N-terminus | N-(acid-PEG₃)- | BCY18004 | C-terminal |
| | | | N-bis(PEG₃-azide) | | Lys(PYA) |
| BCY18048 | BCY9594 | N-terminus | N-(acid-PEG₃)- | BCY17662 | D-Lys(PYA)7 |
| | | | N-bis(PEG₃-azide) | | |
| BCY18049 | BCY9594 | N-terminus | N-(acid-PEG₃)- | BCY18005 | C-terminal |
| | | | N-bis(PEG₃-azide) | | Lys(PYA) |

The heterotandem bicyclic peptide complex BCY15664 consists of an EphA2 specific peptide BCY9594 linked to two NKp46 specific peptides (both of which are BCY15452) via

The heterotandem bicyclic peptide complex BCY15923 consists of an EphA2 specific peptide BCY9594 linked to two NKp46 specific peptides (both of which are BCY 15686) via

The heterotandem bicyclic peptide complex BCY17226 consists of an EphA2 specific peptide BCY9594 linked to two NKp46 specific peptides (both of which are BCY17224) via

The heterotandem bicyclic peptide complex BCY15924 consists of an EphA2 specific peptide BCY9594 linked to two NKp46 specific peptides (both of which are BCY15687) via an N-(acid-PEG₃)-N-bis(PEG₃-azide) linker, shown pictorially as:

The heterotandem bicyclic peptide complex BCY18042 consists of an EphA2 specific peptide BCY9594 linked to two NKp46 specific peptides (both of which are BCY18004) via an N-(acid-PEG₃)-N-bis(PEG₃-azide) linker, shown pictorially as:

The heterotandem bicyclic peptide complex BCY18048 consists of an EphA2 specific peptide BCY9594 linked to two NKp46 specific peptides (both of which are BCY17662) via

The heterotandem bicyclic peptide complex BCY18049 consists of an EphA2 specific peptide BCY9594 linked to two NKp46 specific peptides (both of which are BCY18005) via an N-(acid-PEG₃)-N-bis(PEG₃-azide) linker, shown pictorially as:

TABLE A3
(EphA2:NKp46; 1:3)

| | EphA2 | Attachment | | NKp46 | Attachment |
|---|---|---|---|---|---|
| Complex No. | BCY No. | Point | Linker | BCY No. | Point |
| BCY21686 | BCY9594 | N-terminus | Methane-N- | BCY17224 | C-terminal |
| | | | (PEG₅-acid)- | | Lys(PYA) |
| | | | Tri(MeOPr- | | |
| | | | amide-PEG₄-azide) | | |
| BCY21687 | BCY9594 | N-terminus | Methane-N- | BCY17224 | C-terminal |
| | | | (PEG₁₀-acid)- | | Lys(PYA) |
| | | | Tri(MeOPr- | | |
| | | | amide-PEG₁₀-azide) | | |

The heterotandem bicyclic peptide complex BCY21686 consists of an EphA2 specific peptide BCY9594 linked to three NKp46 specific peptides (all of which are BCY17224) via an Methane-N-(PEG₃-acid)-Tri(MeOPr-amide-PEG₄-azide) linker, shown pictorially as:

The heterotandem bicyclic peptide complex BCY21687 consists of an EphA2 specific peptide BCY9594 linked to three NKp46 specific peptides (all of which are BCY17224) via an Methane-N-(PEG₁₀-acid)-Tri(MeOPr-amide-PEG₁₀-azide) linker, shown pictorially as:

In one specific embodiment, the first peptide ligand comprises an EphA2 binding bicyclic peptide ligand attached to a TATA scaffold, the one or more second peptide ligands comprise two CD16a binding bicyclic peptide ligands attached to a TBMT scaffold and said heterotandem complex is selected from the complexes listed in Table B1:

TABLE B1
(EphA2:CD16a; 1:2)

| | EphA2 | Attachment | | CD16a | Attachment |
|---|---|---|---|---|---|
| Complex No. | BCY No. | Point | Linker | BCY No. | Point |
| BCY15911 | BCY9594 | N-terminus | N-(acid-PEG₃)- | BCY13886 | C-terminal |
| | | | N-bis(PEG₃-azide) | | Lys(PYA) |
| BCY20810 | BCY9594 | N-terminus | N-(acid-PEG₁₀)- | BCY20361 | C-terminal |
| | | | N-bis(PEG₁₀-azide) | | Lys(PYA) |

The heterotandem bicyclic peptide complex BCY15911 consists of an EphA2 specific peptide BCY9594 linked to two CD16a specific peptides (both of which are BCY13886) via

The heterotandem bicyclic peptide complex BCY20810 consists of an EphA2 specific peptide BCY9594 linked to two CD16a specific peptides (both of which are BCY20361) via an N-(acid-PEG₁₀)-N-bis(PEG₁₀-azide) linker, shown pictorially as:

In one specific embodiment, the first peptide ligand comprises an EphA2 binding bicyclic peptide ligand attached to a TATA scaffold, and two second peptide ligands which comprise one NKp46 binding bicyclic peptide ligand attached to a TATA scaffold and one CD16a binding bicyclic peptide ligand attached to a TBMT scaffold and said heterotandem complex is selected from the complexes listed in Table C:

TABLE C
(EphA2:NKp46:CD16a; 1:1:1)

| Complex | EphA2 | Attachment | | NKp46 | Attachment | CD16a | Attachment |
|---|---|---|---|---|---|---|---|
| No. | BCY No. | Point | Linker | BCY No. | Point | BCY No. | Point |
| BCY17231 | BCY9594 | N- | N-(PEG3- | BCY17224 | C- | BCY13883 | C- |
| | | terminus | acid)-N- | | terminal | | terminal |
| | | | (PEG3- | | Lys(PYA) | | Lys(PYA) |
| | | | azide)- | | | | |
| | | | N-(PEG3- | | | | |
| | | | NH-AcAz) | | | | |
| BCY17235 | BCY9594 | N- | TCA- | BCY17224 | C- | BCY13883 | C- |
| | | terminus | [Peg₂₃]₃ | | terminal | | terminal |
| | | | | | Lys(PYA) | | Lys(PYA) |
| BCY20793 | BCY9594 | N- | N-(PEG3- | BCY17224 | C- | BCY20361 | C- |
| | | terminus | acid)-N- | | terminal | | terminal |
| | | | (PEG3- | | Lys(PYA) | | Lys(PYA) |
| | | | azide)- | | | | |
| | | | N-(PEG3- | | | | |
| | | | NH-AcAz) | | | | |

The heterotandem bicyclic peptide complex BCY17231 consists of an EphA2 specific peptide BCY9594 linked to an NKp46 specific peptide (BCY17224) and a CD16a specific peptide (BCY13883) via an N-(PEG₃-acid)-N-(PEG₃-azide)-N-(PEG₃-NH-AcAz) linker, shown pictorially as:

The heterotandem bicyclic peptide complex BCY17235 consists of an EphA2 specific peptide BCY9594 linked to an NKp46 specific peptide (BCY17224) and a CD16a specific peptide (BCY13883) via a TCA-[Peg₂₃]₃ linker, shown pictorially as:

The heterotandem bicyclic peptide complex BCY20793 consists of an EphA2 specific peptide BCY9594 linked to an NKp46 specific peptide (BCY17224) and a CD16a specific peptide (BCY20361) via an N-(PEG₃-acid)-N-(PEG₃-azide)-N-(PEG₃-NH-AcAz) linker, shown pictorially as:

In one specific embodiment, the first peptide ligand comprises an MT1 binding bicyclic peptide ligand attached to a TATA scaffold, the one or more second peptide ligands comprise two NKp46 binding bicyclic peptide ligands attached to a TATA scaffold and said heterotandem complex is selected from the complexes listed in Table D:

TABLE D
(MT1:NKp46; 1:2)

| | MT1 | Attachment | | NKp46 | Attachment |
|---|---|---|---|---|---|
| Complex No. | BCY No. | Point | Linker | BCY No. | Point |
| BCY18604 | BCY14320 | N-terminus | N-(acid-PEG₃)- | BCY17224 | C-terminal |
| | | | N-bis(PEG₃-azide) | | Lys(PYA) |

The heterotandem bicyclic peptide complex BCY18604 consists of an MT1 specific peptide BCY14320 linked to two NKp46 specific peptides (both of which are BCY17224) via an N-(acid-PEG₃)-N-bis(PEG₃-azide) linker, shown pictorially as:

In one specific embodiment, the first peptide ligand comprises an PD-L1 binding bicyclic peptide ligand attached to a TATA scaffold, the one or more second peptide ligands comprise two NKp46 binding bicyclic peptide ligands attached to a TATA scaffold and said heterotandem complex is selected from the complexes listed in Table E:

TABLE E
(PD-L1:NKp46; 1:2)

| | PD-L1 | Attachment | | NKp46 | Attachment |
|---|---|---|---|---|---|
| Complex No. | BCY No. | Point | Linker | BCY No. | Point |
| BCY18603 | BCY11865 | N-terminus | N-(acid-PEG₃)- | BCY17224 | C-terminal |
| | | | N-bis(PEG₃-azide) | | Lys(PYA) |

The heterotandem bicyclic peptide complex BCY18603 consists of a PD-L1 specific peptide BCY11865 linked to two NKp46 specific peptides (both of which are BCY17224) via an N-(acid-PEG₃)-N-bis(PEG₃-azide) linker, shown pictorially as:

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4th ed., John Wiley & Sons, Inc.), which are incorporated herein by reference.

### Nomenclature

### Numbering

When referring to amino acid residue positions within the peptides of the invention, cysteine residues (Cᵢ, Cᵢᵢ and Cᵢᵢᵢ) are omitted from the numbering as they are invariant, therefore, the numbering of amino acid residues within the peptides of the invention is referred to as below:

(SEQ ID NO: 1)
-Cᵢ-N₁-L₂-Q₃-A₄-P₅-Cᵢᵢ-M₆-Q₇-T₈-G₉-K₁₀-V₁₁-Cᵢᵢᵢ-

For the purpose of this description, all bicyclic peptides are assumed to be cyclised with TATA or TBMT and yielding a tri-substituted structure. Cyclisation with TATA or TBMT occurs on the first, second and third reactive groups (i.e. Cᵢ, Cᵢᵢ, Cᵢᵢᵢ).

### Molecular Format

N- or C-terminal extensions to the bicycle core sequence are added to the left or right side of the sequence, separated by a hyphen. For example, an N-terminal BAla-Sar10-Ala tail would be denoted as:

(SEQ ID NO: X)
βAla-Sar10-A-.

### Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa). For the avoidance of doubt, references to amino acids either as their full name or as their amino acid single or three letter codes are intended to be represented herein as L-amino acids unless otherwise stated. If such an amino acid is intended to be represented as a D-amino acid then the amino acid will be prefaced with a lower case d within square parentheses, for example [dA], [dD], [dE], [dK], [d1Nal], [dNle], etc.

### Advantages of the Peptide Ligands

The multifunctional NK cell engager (NKCE) approach combines and enhances NK cell immune surveillance capabilities as well as their cytotoxic function. Bi-specific (BIKE), tri-specific (TRIKE), or tetra-specific killer engagers (TetraKE) are small, engineered antibody molecules designed to create a connection between the effector NK cell and the targeted tumour cells. (Felices et al. 2016, Moore et al. 2011). Importantly, mAb-mediated activation of NCRs results in an activation of NK cytotoxicity against many types of target cells. The cross-linking, induced by the specific mAbs, leads to a strong NK cell activation resulting in increased cytotoxicity, and cytokine production. These engagers contain an anti-CD16 antibody, which will bind CD16 to trigger NK cell cytotoxicity, and an antibody or antigen for the tumour cell. An example of BiKE is CD16×CD33 which enhances the NK activity against CD33+HL60 AML cell line in vitro (Gleason et al. 2014). TRIKE and TetraKE use the cytokine interleukin 15 (IL-15) molecule as a link between the antibodies, exhibiting more cytotoxicity and generation of inflammatory cytokines than BiKEs (Davis et al. 2017, Felices et al. 2019). Seeking to maximize NK anti-tumor response, the Vivier group recently showed the potency of a multifunctional NKCE composed of the Fc fragment for CD16 binding, as well as two antibody domains targeting the activating NK cell receptor, NKp46, and a specific tumor antigen, such as CD19, CD20, and EGFR (Gauthier et al. 2019). This NKCE demonstrated enhanced NK cell infiltration into tumors and promoted tumor clearance in in vivo models, and further illustrated enhanced efficacy over the current antibodies in clinical use, such as rituximab and cetuximab. Moreover, harnessing the activating potential of multiple stimulatory receptors (CD16 and NKp46) on the NK cell, inhibition was overcome and full NK cell activity achieved (BenShumel 2020, Tarzona 2020, Davis et al. 2017).

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should in most circumstances demonstrate stability to plasma proteases, epithelial (“membrane-anchored”) proteases, gastric and intestinal proteases, lung surface proteases, intracellular proteases and the like. Protease stability should be maintained between different species such that a bicyclic peptide lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes; and
An optimal plasma half-life in the circulation. Depending upon the clinical indication and treatment regimen, it may be required to develop a bicyclic peptide with short or prolonged in vivo exposure times for the management of either chronic or acute disease states. The optimal exposure time will be governed by the requirement for sustained exposure (for maximal therapeutic efficiency) versus the requirement for short exposure times to minimise toxicological effects arising from sustained exposure to the agent.

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three reactive groups selected from cysteine, 3-mercaptopropionic acid and/or cysteamine and form at least two loops on the scaffold.

### Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a lysine side chain, or an N-terminal amine group or any other suitable reactive group, such as penicillamine. Details of suitable reactive groups may be found in WO 2009/098450.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, a polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and l and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis et al., Nat Chem Biol 2009, 5 (7), 502-7.

In one embodiment, the reactive groups are selected from cysteine, 3-mercaptopropionic acid and/or cysteamine residues.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (+)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the invention contain an amine function, these may form quaternary ammonium salts, for example by reaction with an alkylating agent according to methods well known to the skilled person. Such quaternary ammonium compounds are within the scope of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, C□-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise ß-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the Nectin-4 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment, the molecular scaffold may be a macromolecule. In one embodiment, the molecular scaffold is a macromolecule composed of amino acids, nucleotides or carbohydrates.

In one embodiment, the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene (the scaffold reactive group exemplified by TBMB) or iodoacetamide. Other scaffold reactive groups that are used to selectively couple compounds to cysteines in proteins are maleimides, α-unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene. An example of an □□ unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606). An example of an α-halomethylcarbonyl containing compound is N,N′, N″-(benzene-1,3,5-triyl)tris(2-bromoacetamide). Selenocysteine is also a natural amino acid which has a similar reactivity to cysteine and can be used for the same reactions. Thus, wherever cysteine is mentioned, it is typically acceptable to substitute selenocysteine unless the context suggests otherwise.

In one embodiment, the molecular scaffold is 1,1′,1″-(1,3,5-triazinane-1,3,5-triyl)triprop-2-en-1-one (also known as triacryloylhexahydro-s-triazine (TATA):

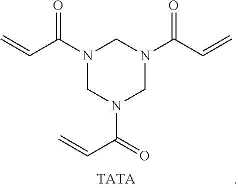

Thus, following cyclisation with the bicyclic peptides of the invention on the Cᵢ, Cᵢᵢ, and Cᵢᵢ cysteine residues, the molecular scaffold forms a tri-substituted 1,1′,1″-(1,3,5-triazinane-1,3,5-triyl) tripropan-1-one derivative of TATA having the following structure:

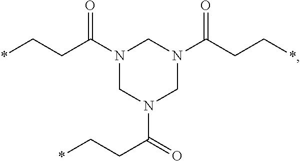

wherein * denotes the point of attachment of the three cysteine residues.

In an alternative embodiment, the molecular scaffold is 2,4,6-tris(bromomethyl)-s-triazine (TBMT):

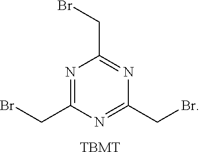

Thus, following cyclisation with the bicyclic peptides of the invention on the Cᵢ, Cᵢᵢ, and Cᵢᵢᵢ cysteine residues, the molecular scaffold forms a tri-substituted derivative of TBMT having the following structure:

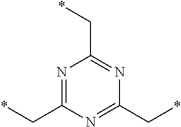 [SMILES: *Cc1nc(C*)nc(C*)n1]

wherein * denotes the point of attachment of the three cysteine residues.

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptides to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptides, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

According to a further aspect of the invention, there is provided a heterotandem bicyclic peptide complex as defined herein for use in preventing, suppressing or treating cancer.

Examples of cancers (and their benign counterparts) which may be treated (or inhibited) include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumors of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcomaprotuberans; tumors of the central or peripheral nervous system (for example astrocytomas, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and paediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).

In a further embodiment, the cancer is selected from a hematopoietic malignancy such as selected from: non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), and chronic myeloid leukemia (CML).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

In general, some of the heterotandem bicyclic peptide complexes of the invention may be prepared in accordance with the following general method:

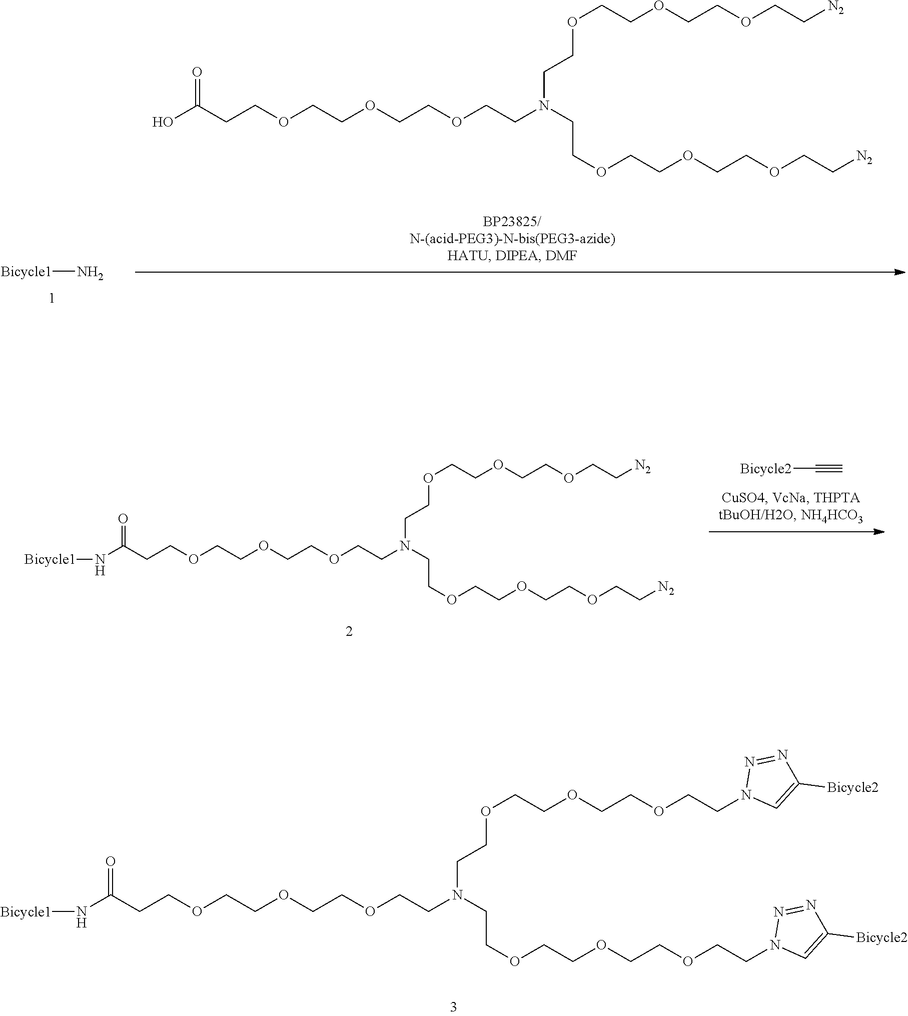

All solvents are degassed and purged with N₂. A solution of BP23825 (1.0 eq), HATU (1.2 eq) and DIPEA (2.0 eq) in DMF is mixed for 5 minutes, then Bicycle1 (1.2 eq) is added. The reaction mixture is stirred at 40° C. for 16 hr. The reaction mixture is then concentrated under reduced pressure to remove solvent and purified by prep-HPLC to give intermediate 2.

A mixture of intermediate2 (1.0 eq) and Bicycle2 (2.0 eq) are dissolved in t-BuOH/H₂O (1:1), and then CuSO₄ (1.0 eq), VcNa (4.0 eq), and THPTA (2.0 eq) are added. Finally, 0.2 M NH₄HCO₃ is added to adjust pH to 8. The reaction mixture is stirred at 40° C. for 16 hr under N₂ atmosphere. The reaction mixture was directly purified by prep-HPLC.

Heterotandem bicyclic peptide complexes which were prepared using this method are listed below:

| EphA2/NKp46 | EphA2/CD16a | PD-L1/NKp46 | MT1/NKp46 |
|---|---|---|---|
| BCY15664, BCY15923, | BCY15911 | BCY18603 | BCY18604 |
| BCY17226, BCY15924, | | | |
| BCY18042, BCY18048, | | | |
| BCY18049 | | | |

More detailed experimental for selected heterotandem bicyclic peptide complexes of the invention are provided herein below:

### Example 1: Synthesis of Intermediate BP23825-BCY9594

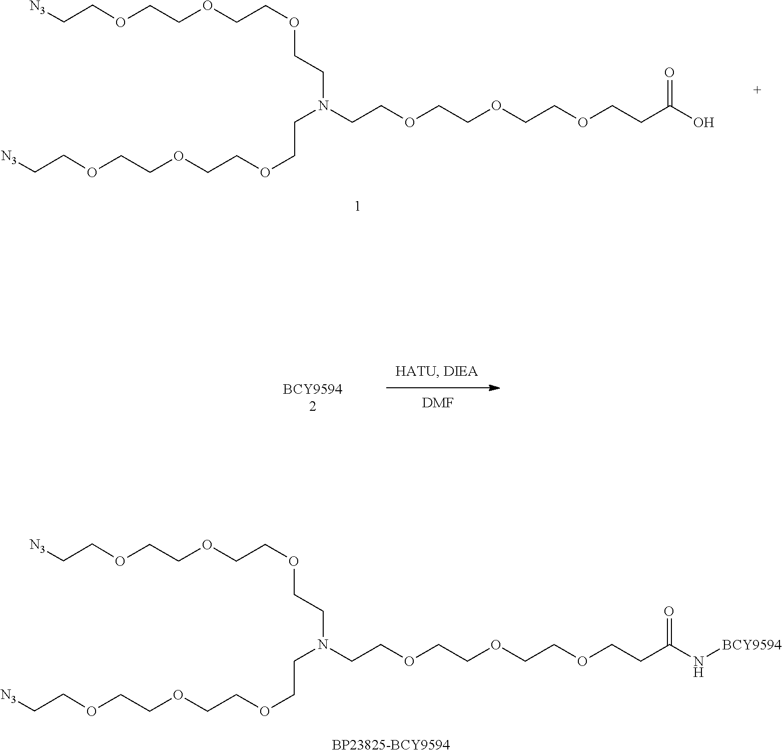

To a mixture of compound 1 (BP23825, 60.0 mg, 96.2 μmol, 1.0 eq) in DMF (3 mL) was added DIEA (12.4 mg, 96.2 μmol, 16.8 μL, 1.0 eq) and HATU (38.4 mg, 101 μmol, 1.05 eq) and the mixture stirred for 5 min. Then BCY9594 (243 mg, 101 μmol, 1.05 eq) was added to the mixture, which was purged with N₂, then stirred at 40° C. for 16 hr under N₂ atmosphere. LC-MS showed compound 1 was consumed completely and one main peak with desired m/z was detected. The reaction mixture was purified by preparative-HPLC to give BP23825-BCY9594 (154 mg, 48.1 μmol, 50.0% yield, 94.0% purity) as a white solid. Calculated MW: 3006.48, observed m/z: 1002.8 ([M+3H]³⁺), 1504.4 ([M+2H]²⁺).

### Example 2: Synthesis of BCY15664

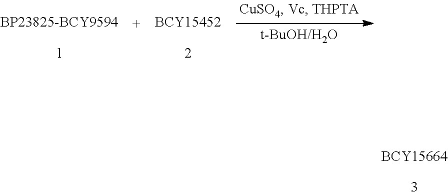

A mixture of compound 1 (20.0 mg, 6.65 μmol, 1.0 eq.), compound 2 (30.0 mg, 13.97 μmol, 2.1 eq.), and THPTA (2.9 mg, 6.65 μmol, 1.0 eq.) was dissolved in t-BuOH/H₂O (1:1, 1 mL), which had been degassed and purged with N₂. An aqueous solution of CuSO₄ (0.4 M, 16.6 μL, 1.0 eq.) and Vc (4.7 mg, 26.61 μmol, 4.0 eq.) were added under N₂. The pH of this solution was adjusted to 8 by dropwise addition of 0.2 M NH₄HCO₃ (in 1:1 t-BuOH/H₂O), and the solution turned light yellow. The reaction mixture was stirred at 25° C. for 1 hr under an N₂ atmosphere. LC-MS showed BP23825-BCY9594 was consumed completely, and one main peak with desired m/z was detected. The reaction mixture was filtered to remove undissolved residue. The crude product was purified by preparative HPLC, and BCY15664 (15.0 mg, 1.97 μmol, 29.6% yield, 97.7% purity) was obtained as a white solid. Calculated MW: 7317.3, observed m/z: 1220.3 ([M+6H]⁶⁺), 915.5 ([M+8H]⁸⁺).

### Example 3: Synthesis of BCY15911

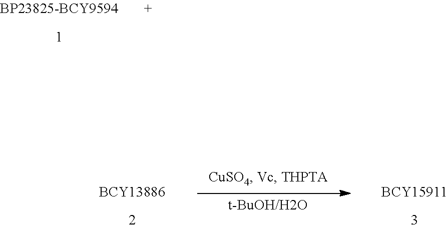

A mixture of compound 1 (20.0 mg, 6.65 μmol, 1.0 eq.), compound 2 (31.6 mg, 16.63 μmol, 2.5 eq.), and THPTA (2.9 mg, 6.65 μmol, 1.0 eq.) was dissolved in t-BuOH/H₂O (1:1, 1 mL), which had been degassed and purged with N₂. An aqueous solution of CuSO₄ (0.4 M, 16.6 μL, 1.0 eq.) and Vc (4.7 mg, 26.61 μmol, 4.0 eq.) were added under N₂. The pH of this solution was adjusted to 8, and the solution turned light yellow. The reaction mixture was stirred at 25° C. for 1 hr under an N₂ atmosphere. LC-MS showed BP23825-BCY9594 was consumed completely, and one main peak with desired m/z was detected. The reaction mixture was filtered to remove undissolved residue. The crude product was purified by preparative HPLC and BCY15911 (10.4 mg, 1.50 μmol, 22.61% yield, 94% purity) was obtained as a white solid. Calculated MW: 6814.8, observed m/z: 1364.0 ([M+5H]⁵⁺), 1137.0 ([M+6H]⁶⁺).

### Example 4: Synthesis of BCY15923

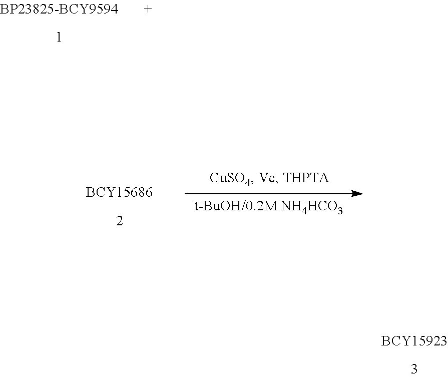

A mixture of compound 1 (20.0 mg, 6.65 μmol, 1.0 eq.), compound 2 (35.0 mg, 16.79 μmol, 2.5 eq.), and THPTA (2.9 mg, 6.65 μmol, 1.0 eq.) was dissolved in t-BuOH/0.2 M NH₄HCO₃ (1:1, 1 mL), pre-degassed and purged with N₂ for 3 times, and then an aqueous solution of CuSO₄ (0.4 M, 16.6 μL, 1.0 eq.) and ascorbic acid (4.69 mg, 26.61 μmol, 4 eq.) were added under N₂ atmosphere. The pH of this solution was adjusted to 8 by dropwise addition of 0.2 M NH₄HCO₃, and the solution turned to light yellow. The reaction mixture was stirred at 25° C. for 2 hr under N₂ atmosphere. LC-MS showed compound 1 was consumed completely, and desired m/z (calculated MW: 7191.31, observed m/z: 1439.3 ([M/5+H]⁺), 1199.6 ([M/6+H]⁺)) was detected. The reaction mixture was filtered to remove the insoluble residue. The crude product was purified by prep-HPLC (TFA condition), and BCY15923 (4 mg, 5.28e-1 μmol, 7.94% yield, 95% purity) was obtained as a white solid.

### Example 5: Synthesis of BCY17226

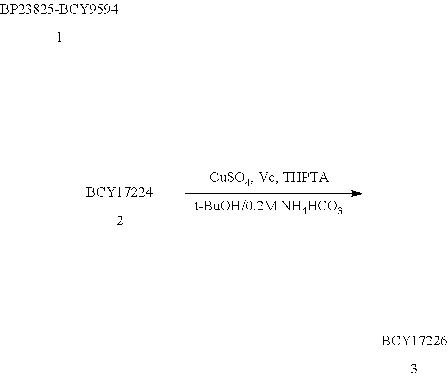

A mixture of compound 1 (20.0 mg, 6.65 μmol, 1.0 eq.), compound 2 (30.5 mg, 14.64 μmol, 2.2 eq.), and THPTA (5.8 mg, 13.30 μmol, 2.0 eq.) was dissolved in t-BuOH/H₂O (1:1, 1.0 mL, pre-degassed and purged with N₂ for 3 times), and then aqueous solution of CuSO₄ (0.4 M, 24.9 μL, 1.5 eq.) and VcNa (4.0 mg, 19.96 μmol, 3.0 eq.) were added under N₂ atmosphere. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 0.5 hr under N₂ atmosphere. LC-MS showed compound 1 was consumed completely, and one main peak containing desired m/z (calculated MW: 7169.18, observed m/z: 1434.9 ([M/5+H]⁺), 1195.9 ([M/6+H]⁺)) was detected. The reaction mixture was filtered to remove the undissolved residue. The crude product was purified by prep-HPLC (TFA condition), and BCY17226 (24.0 mg, 3.35 μmol, 50.32% yield. 97.0% purity) was obtained as a white solid.

### Example 6: Synthesis of BCY15924

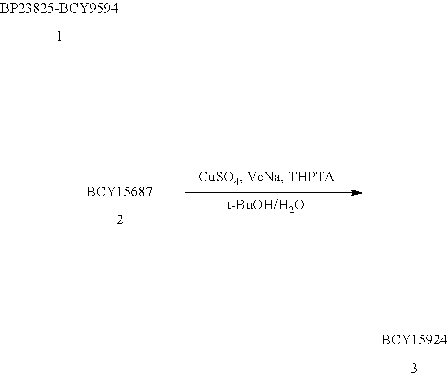

A mixture of compound 1 (5 mg, 1.66 μmol, 1.0 eq), compound 2 (6.97 mg, 3.33 μmol, 2.0 eq), and THPTA (1.45 mg, 3.33 μmol, 2.0 eq) was dissolved in t-BuOH/H₂O (1:1, 2.0 mL, pre-degassed and purged with N₂ for 3 times), and then aqueous solution of CuSO₄ (0.4 M, 6.24 μL, 1.5 eq) and VcNa (988.40 μg, 4.99 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 1 was consumed completely, and one main peak with desired m/z (calculated MW: 7195.55, observed m/z: 1440.1 ([M/5+H]⁺), 1200.2 ([M/6+H]⁺), 1029.1 ([M/7+H]⁺)) was detected. The reaction mixture was filtered to remove the undissolved residue. The crude product was purified by prep-HPLC (TFA condition), and BCY15924 (3.4 mg, 4.52e-1 μmol, 27.16% yield, 95.6% purity) was obtained as a white solid.

### Example 7: Synthesis of BCY18042

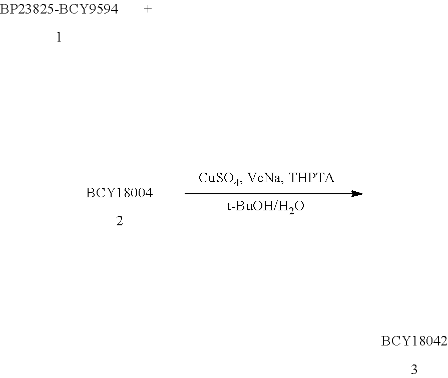

A mixture of compound 1 (15 mg, 4.99 μmol, 1.0 eq), compound 2 (28.08 mg, 10.48 μmol, 2.1 eq), and THPTA (4.34 mg, 9.98 μmol, 2.0 eq) was dissolved in t-BuOH/H₂O (1:1, 2.0 mL, pre-degassed and purged with N₂ for 3 times), and then aqueous solution of CuSO₄ (0.4 M, 18.71 UL, 1.5 eq) and VcNa (2.97 mg, 14.97 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 1 was consumed completely, and one main peak with desired m/z (calculated MW: 6999.35, observed m/z: 1400.7 ([M/5+H]+), 1167.6 ([M/6+H]+), 1000.9 ([M/7+H]+)) was detected. The reaction mixture was filtered to remove the undissolved residue. The crude product was purified by prep-HPLC (TFA condition), and BCY18042 (3.9 mg, 5.15e-1 μmol, 10.32% yield, 96.0% purity) was obtained as a white solid.

### Example 8: Synthesis of BCY18048

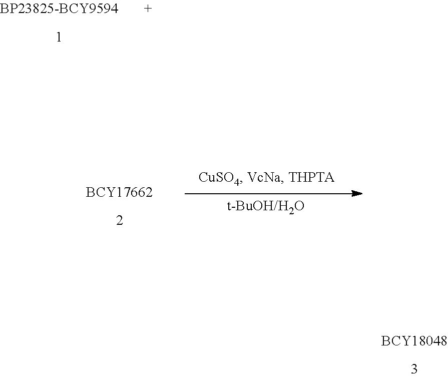

A mixture of compound 1 (15 mg, 4.99 μmol, 1.0 eq), compound 2 (21.99 mg, 10.48 μmol, 2.1 eq), and THPTA (4.34 mg, 9.98 μmol, 2.0 eq) was dissolved in t-BuOH/H₂O (1:1, 2.0 mL, pre-degassed and purged with N₂ for 3 times), and then aqueous solution of CuSO₄ (0.4 M, 25.0 μL, 2.0 eq) and VcNa (2.97 mg, 14.97 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 1 was consumed completely, and one main peak with desired m/z (calculated MW: 7203.30, observed m/z: 1441.3 ([M/5+H]⁺), 1201.3 ([M/6+H]⁺), 901.7 ([M/8+H]⁺)) was detected. The reaction mixture was filtered to remove the undissolved residue. The crude product was purified by prep-HPLC (TFA condition), and BCY18048 (7.4 mg, 9.85e-1 μmol, 19.74% yield, 97.4% purity) was obtained as a white solid.

### Example 9: Synthesis of BCY18049

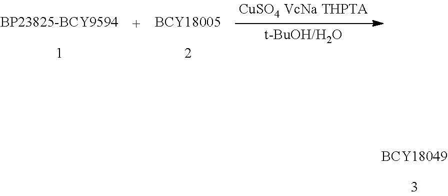

A mixture of compound 1 (15 mg, 4.99 μmol, 1.0 eq), compound 2 (24.41 mg, 10.48 μmol, 2.1 eq), and THPTA (4.34 mg, 9.98 μmol, 2.0 eq) was dissolved in t-BuOH/H₂O (1:1, 2.0 mL, pre-degassed and purged with N₂ for 3 times), and then aqueous solution of CuSO₄ (0.4 M, 18.71 μL, 1.5 eq) and VcNa (2.97 mg, 14.97 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 1 was consumed completely, and one main peak with desired m/z (calculated MW: 7666.02, observed m/z: 959.3 ([M/8+H]⁺), 1096.2 ([M/7+H]⁺), 1278.6 ([M/6+H]⁺)) was detected. The reaction mixture was filtered to remove the undissolved residue. The crude product was purified by prep-HPLC (TFA condition), and BCY18049 (10.6 mg, 1.37 μmol, 27.41% yield, 98.9% purity) was obtained as a white solid.

### Example 10: Synthesis of BCY18603

Preparation of Intermediate BP23825-BCY11865

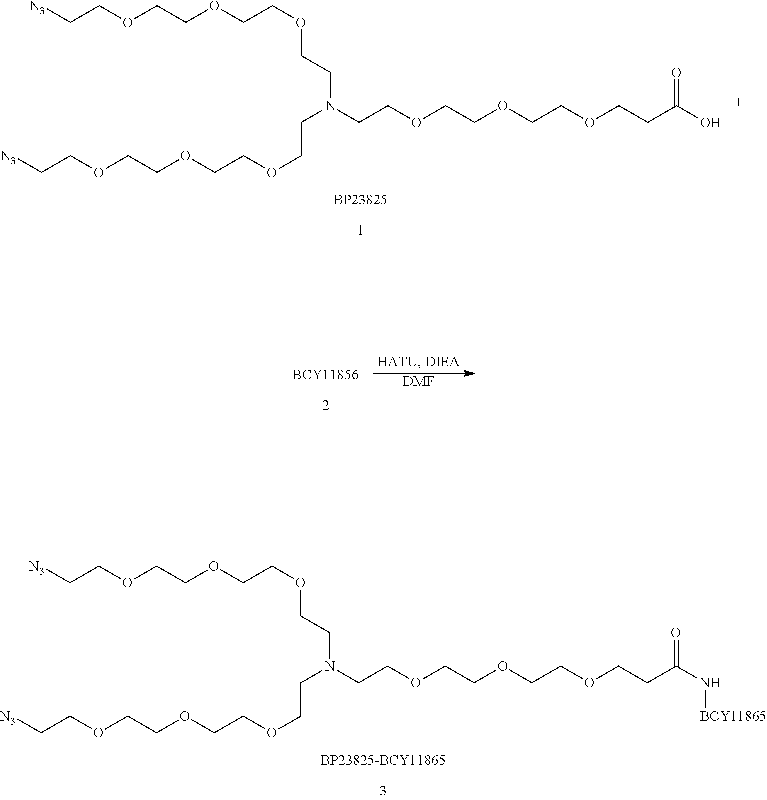

A mixture of compound 1 (15.0 mg, 24.0 μmol, 1.0 eq), HATU (10.1 mg, 26.5 μmol, 1.1 eq) and DIEA (9.3 mg, 72.1 μmol, 12.6 μL, 3.0 eq) was dissolved in DMF (1.0 mL). The reaction mixture was activated at 25-30° C. for 6 min, then compound 2 (68.5 mg, 26.5 μmol, 1.1 eq) was added into the reaction mixture. The reaction mixture was stirred at 25-30° C. for 0.5 hr. LC-MS showed one main peak with desired m/z (MW: 3196.7, observed m/z: 1066.6 [M/3+H]⁺, 800.2 [M/4+H]⁺) was detected. The reaction mixture was filtered to remove the insoluble residue. The soluble crude product was then purified by prep-HPLC (TFA condition). Compound 3 (BP23825-BCY11865, 43.0 mg, 13.4 μmol, 55.8% yield, 97.4% purity) was obtained as a white solid.

Preparation of BCY18603

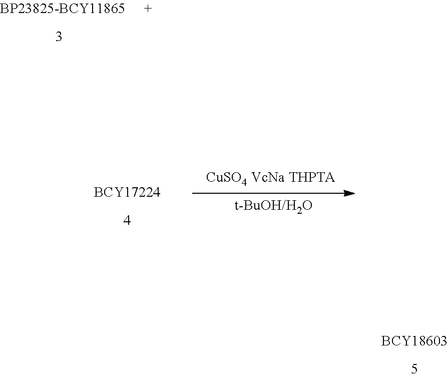

A mixture of compound 3 (15.0 mg, 4.69 μmol, 1.0 eq), compound 4 (20.5 mg, 9.85 μmol, 2.1 eq) and THPTA (2.0 mg, 4.69 μmol, 1.0 eq) was dissolved in t-BuOH/H₂O (1:1, 2.0 mL, pre-degassed and purged with N₂ for 3 times), and then aqueous solution of CuSO₄ (0.4 M, 17.6 μL, 1.5 eq) and VcNa (2.8 mg, 14.1 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 3 was consumed completely, and one main peak with desired m/z (calculated MW: 7359.3, observed m/z: 1472.9 [M/5+H]⁺, 1227.5 [M/6+H]⁺) was detected. The reaction mixture was filtered to remove the insoluble residue.

The crude product was purified by prep-HPLC (TFA condition), and BCY18603 (13.4 mg, 1.82 μmol, 38.8% yield, 97.8% purity) was obtained as a white solid.

### Example 11: Synthesis of BCY18604

Preparation of Intermediate BP23825-BCY14320

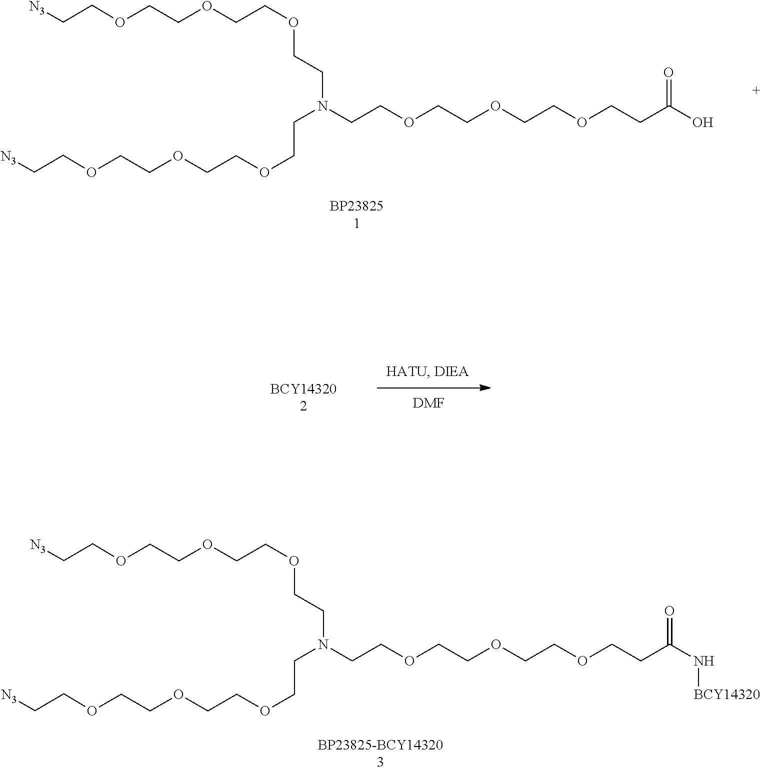

A mixture of compound 1 (20.0 mg, 32.1 μmol, 1.0 eq), HATU (13.4 mg, 35.3 μmol, 1.1 eq) and DIEA (12.4 mg, 96.2 μmol, 16.8 μL, 3.0 eq) was dissolved in DMF (1.0 mL). The reaction mixture was activated at 25-30° ° C. for 6 min, then compound 2 (74.4 mg, 35.3 μmol, 1.1 eq) was added into the reaction mixture. The reaction mixture was stirred at 25-30° C. for 0.5 hr. LC-MS showed one main peak with desired m/z (MW: 2714.27, observed m/z: 1358.1 [M/2+H]⁺, 905.9 [M/3+H]⁺, 679.5 [M/4+H]⁺) was detected. The reaction mixture was filtered to remove the undissolved residue. The residue was then purified by prep-HPLC (TFA condition). Compound 3 (BP23825-BCY14320, 42.3 mg, 15.6 μmol, 48.5% yield, 97.7% purity) was obtained as a white solid.

Preparation of BCY18604

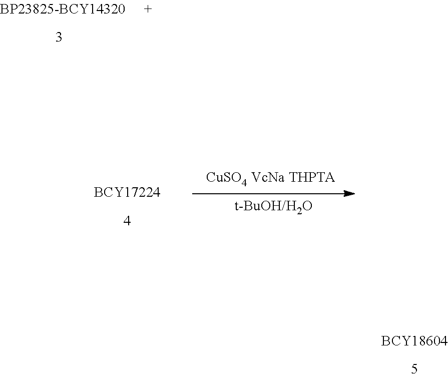

A mixture of compound 3 (15.0 mg, 5.53 μmol, 1.0 eq), compound 4 (24.2 mg, 11.6 μmol, 2.1 eq) and THPTA (2.4 mg, 5.53 μmol, 1.0 eq) was dissolved in t-BuOH/H₂O (1:1, 2.0 mL, pre-degassed and purged with N₂ for 3 times), and then aqueous solution of CuSO₄ (0.4 M, 20.7 μL, 1.5 eq) and VcNa (3.3 mg, 16.6 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° ° C. for 1 hr under N₂ atmosphere. LC-MS showed compound 3 was consumed completely, and one main peak with desired m/z (calculated MW: 6876.9, observed m/z: 1376.4 [M/5+H]⁺, 1147.3 [M/6+H]⁺) was detected. The reaction mixture was filtered to remove the insoluble residue. The crude product was purified by prep-HPLC (TFA condition), and BCY18604 (19.1 mg, 2.77 μmol, 50.0% yield, 97.1% purity) was obtained as a white solid.

### Example 12: Synthesis of BCY20810

Preparation of BP25635-BCY9594

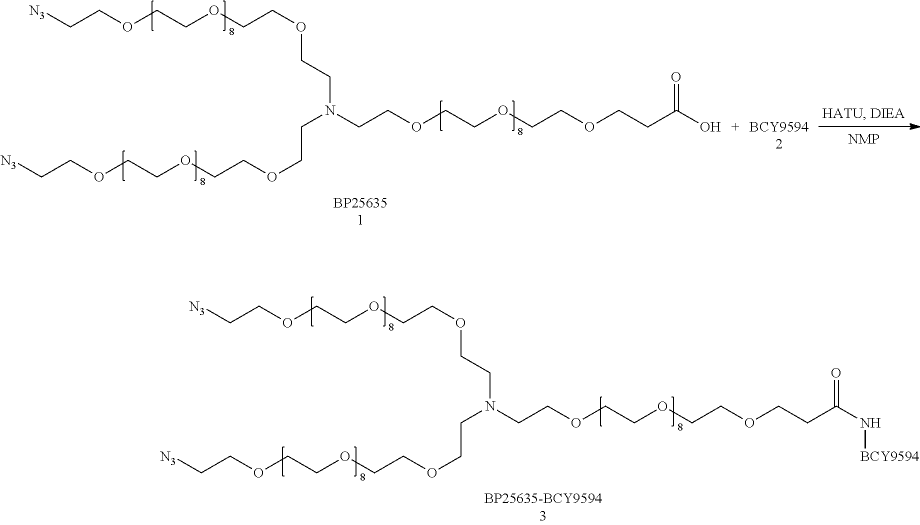

A mixture of compound 1 (50.0 mg, 32.3 μmol, 1.0 eq.), HATU (14.7 mg, 38.7 μmol, 1.2 eq) and DIEA (8.3 mg, 64.5 μmol, 11.2 μL, 2.0 eq.) was dissolved in NMP (0.5 mL). The reaction mixture was activated at 25-30° C. for 5 min, then compound 2 (85.2 mg, 35.5 μmol, 1.1 eq.) was added into the reaction mixture. The reaction mixture was stirred at 25-30° C. for 0.5 hr. LC-MS showed one main peak with desired m/z (MW:3931.5, observed m/z: 1310.5 [M/3+H]⁺, 983.8 [M/4+H]⁺) was detected. The reaction mixture was filtered to remove the insoluble residue. The soluble crude product was then purified by prep-HPLC (TFA condition). Compound 3 (BP25635-BCY9594, 34.6 mg, 8.80 μmol, 27.2% yield, 96.8% purity) was obtained as a white solid.

Preparation of BCY20810

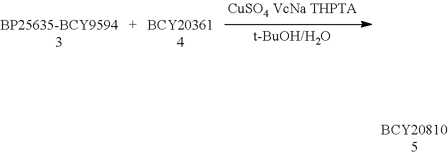

A mixture of compound 3 (15.0 mg, 3.82 μmol, 1.0 eq), compound 4 (15.6 mg, 7.63 μmol, 2.0 eq), and THPTA (3.3 mg, 7.63 μmol, 2.0 eq) was dissolved in t-BuOH/H₂O (1:1, 0.3 mL, pre-degassed and purged with N₂ for 3 times), and then aqueous solution of CuSO₄ (0.4 M, 14.3 UL, 1.5 eq) and VcNa (2.3 mg, 11.4 μmol, 3.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 0.5 hr under N₂ atmosphere. LC-MS showed compound 3 was consumed completely, and one main peak with desired m/z (calculated MW: 8028.3, observed m/z: 1338.7 [M/6+H]⁺, 1147.7 [M/7+H]⁺, 1004.4 [M/8+H]⁺) was detected. The reaction mixture was filtered to remove the insoluble residue. The soluble crude product was purified by prep-HPLC (TFA condition), and BCY20810 (3.0 mg, 3.73e-1 μmol, 9.76% yield, 95.2% purity) was obtained as a white solid.

### Example 13: Synthesis of BCY21686

Preparation of Intermediate BP25517-BCY9594

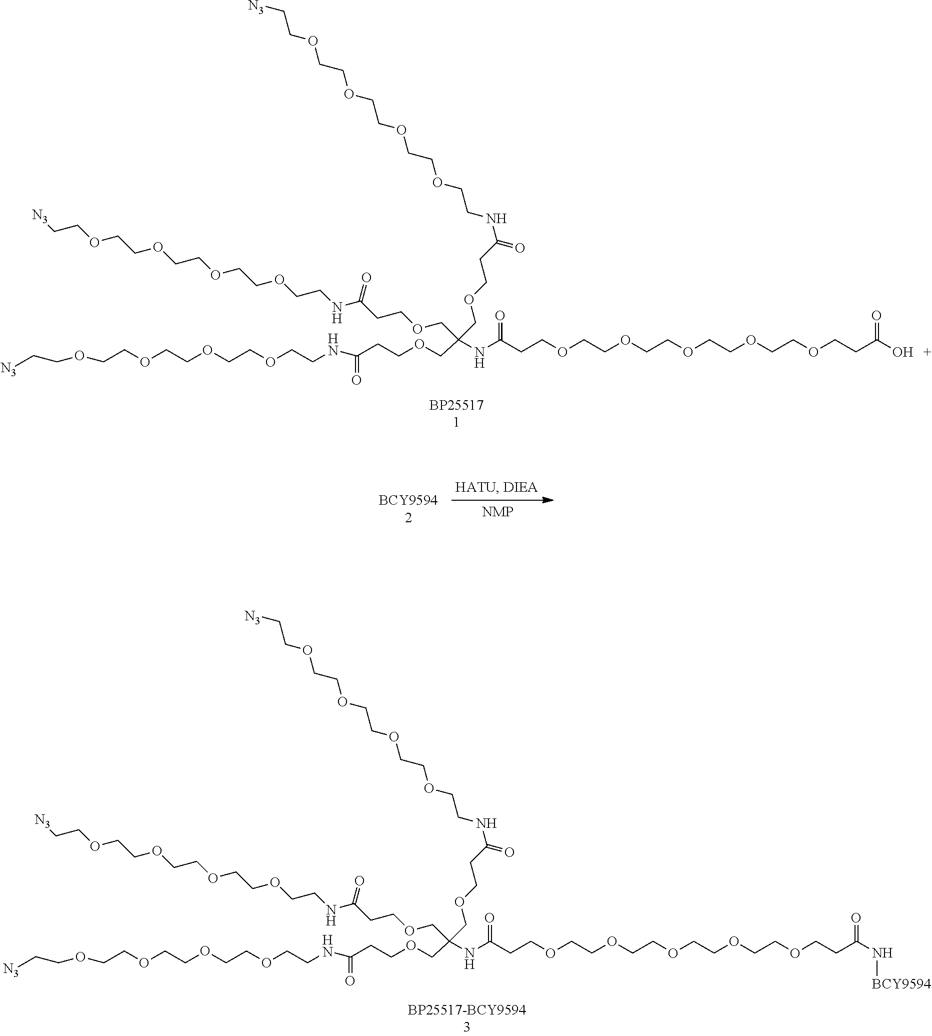

A mixture of compound 1 (10.0 mg, 7.19 μmol, 1.0 eq.), HATU (3.3 mg, 8.6 μmol, 1.2 eq) and DIEA (2.8 mg, 21.6 μmol, 3.8 μL, 3.0 eq.) was dissolved in NMP (0.5 mL). The reaction mixture was activated at 25-30° C. for 5 min, then compound 2 (19.0 mg, 7.91 μmol, 1.1 eq) was added into the reaction mixture. The reaction mixture was stirred at 25-30° C. for 0.5 hr. LC-MS showed one main peak with desired m/z (calculated MW:3773.3, observed m/z: 1258.4 [M/3+H]⁺, 944.1 [M/4+H]⁺, 755.5 [M/5+H]⁺) was detected. The reaction mixture was filtered to remove the undissolved residue. The crude product was then purified by prep-HPLC (TFA condition). Compound 3 (BP25517-BCY9594, 9.5 mg, 2.52 μmol, 35.0% yield, 95.3% purity) was obtained as a white solid.

Preparation of BCY21686

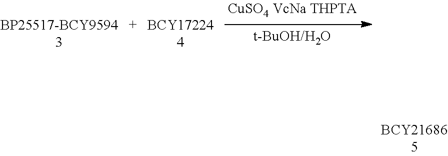

A mixture of compound 3 (9.5 mg, 2.52 μmol, 1.0 eq), compound 4 (15.7 mg, 7.55 μmol, 3.0 eq), and THPTA (2.2 mg, 5.04 μmol, 2.0 eq) was dissolved in t-BuOH/H₂O (1:1, 0.3 mL, pre-degassed and purged with N₂ for 3 times), and then aqueous solution of CuSO₄ (0.4 M, 12.6 μL, 2.0 eq) and VcNa (2.0 mg, 10.0 μmol, 4.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 0.5 hr under N₂ atmosphere. LC-MS showed compound 3 was consumed completely, and one main peak with desired m/z (calculated MW: 10017.3, observed m/z: 1431.7 [M/7+H]⁺, 1252.9 [M/8+H]⁺) was detected. The reaction mixture was filtered to remove the undissolved residue. The crude product was purified by prep-HPLC (TFA condition), and BCY21686 (12.8 mg, 1.23 μmol, 48.8% yield, 96.4% purity) was obtained as a white solid.

### Example 14: Synthesis of BCY21687

Preparation of Intermediate BP25668-BCY9594

A mixture of compound 1 (15.0 mg, 6.24 μmol, 1.0 eq), HATU (2.8 mg, 7.49 μmol, 1.2 eq) and DIEA (2.4 mg, 18.7 μmol, 3.3 L, 3.0 eq.) was dissolved in NMP (0.5 mL). The reaction mixture was activated at 25-30° ° C. for 5 min, then compound 2 (16.5 mg, 6.86 μmol, 1.1 eq) was added into the reaction mixture. The reaction mixture was stirred at 25-30° C. for 0.5 hr. LC-MS showed one main peak with desired m/z (MW:4786.5, observed m/z:1197.4 [M/4+H]⁺) was detected. The reaction mixture was filtered to remove the undissolved residue. The crude product was then purified by prep-HPLC (TFA condition). Compound 3 (BP25668-BCY9594, 13.6 mg, 2.84 μmol, 45.51% yield, 95.5% purity) was obtained as a white solid.

Preparation of BCY21687

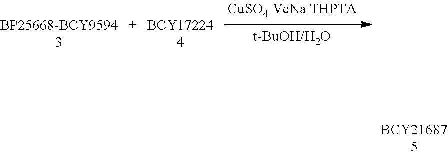

A mixture of compound 3 (13.6 mg, 2.84 μmol, 1.0 eq), compound 4 (17.3 mg, 8.32 μmol, 3.0 eq), and THPTA (2.5 mg, 5.68 μmol, 2.0 eq) was dissolved in t-BuOH/H₂O (1:1, 0.3 mL, pre-degassed and purged with N₂ for 3 times), and then aqueous solution of CuSO₄ (0.9 mg, 5.68 μmol, 2.0 eq) and VcNa (2.2 mg, 11.37 μmol, 4.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 0.5 hr under N₂ atmosphere. LC-MS showed compound 3 was consumed completely, and one main peak with desired m/z (calculate MW: 11030.5, observed m/z: 1379.6 [M/8+H]⁺, 1226.5 [M/9+H]⁺) was detected. The reaction mixture was filtered to remove the insoluble residue.

The soluble crude product was purified by prep-HPLC (TFA condition), and BCY21687 (14.5 mg, 1.31 μmol, 46.1% yield, 94.5% purity) was obtained as a white solid.

### Examples 15: Synthesis of BCY17231

Preparation of Intermediate BP24527-BCY9594

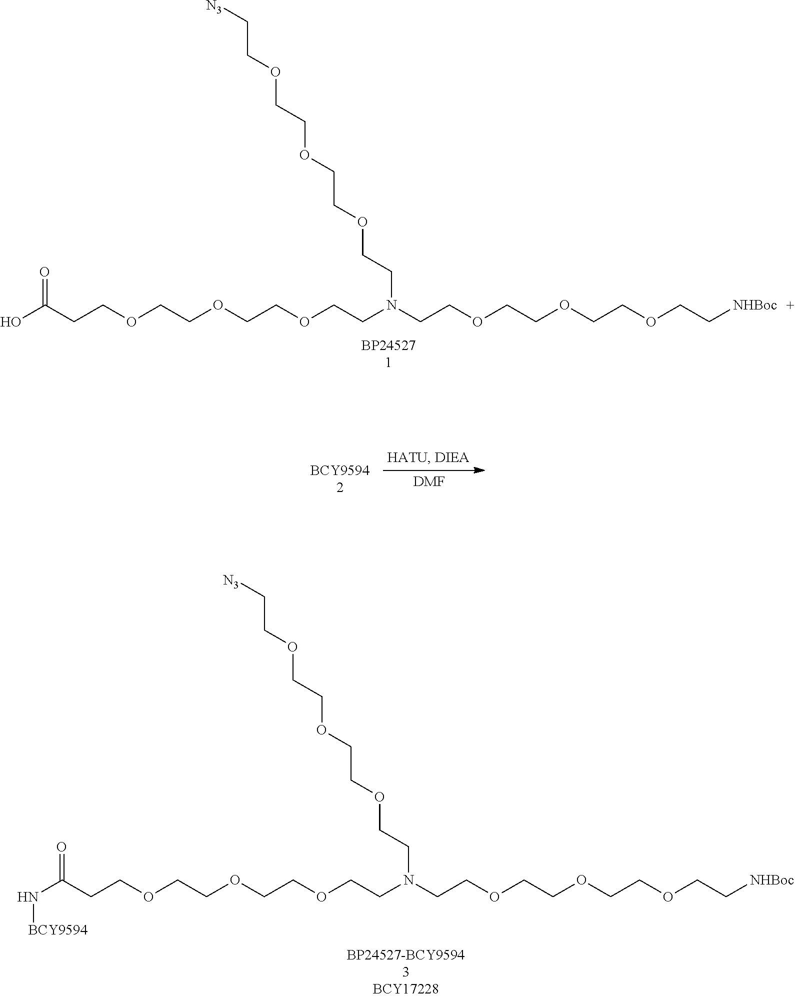

A mixture of compound 1 (100.0 mg, 143 μmol, 1.0 eq.), HATU (59.9 mg, 157.6 μmol, 1.1 eq) and DIEA (55.56 mg, 429.9 μmol, 74.88 μL, 3.0 eq.) was dissolved in DMF (5.0 mL). The reaction mixture was activated at 25-30° C. for 6 min, then compound 2 (378 mg, 157 μmol, 1.1 eq.) was added into the reaction mixture. The reaction mixture was stirred at 25-30° C. for 0.5 hr. LC-MS showed one main peak with desired m/z (MW: 3080.59, observed m/z: 1027.9[(M/3+H]⁺, 771.2 [(M/4+H]⁺) was detected. The reaction mixture was filtered to remove the insoluble residue. The residue was then purified by prep-HPLC (TFA condition). Compound 3 (BP24527-BCY9594, 342 mg, 111.02 μmol, 77.4% yield, 91.5% purity) was obtained as a white solid.

Preparation of Intermediate BCY17230

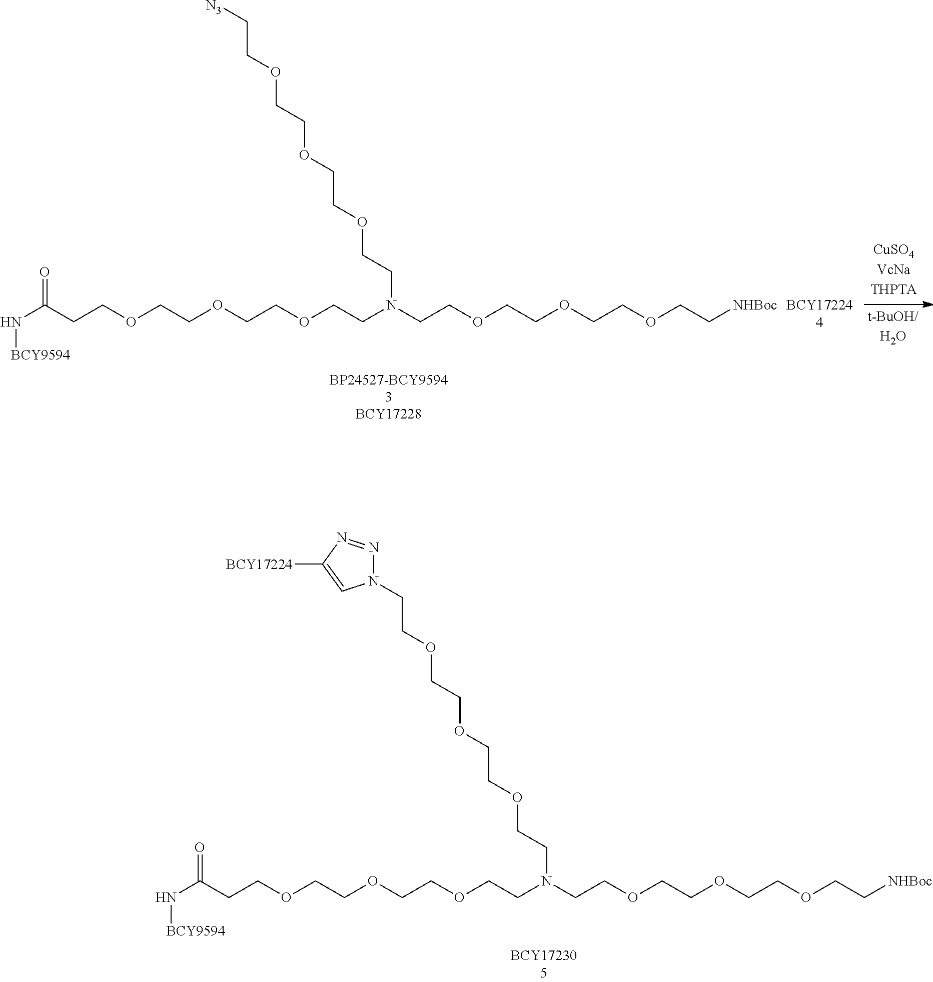

A mixture of compound 3 (100.0 mg, 32.5 μmol, 1.0 eq), compound 4 (74.3 mg, 35.7 μmol, 1.1 eq), and THPTA (14.1 mg, 32.5 μmol, 1.0 eq) was dissolved in t-BuOH/H₂O (1:1, 4.0 mL, pre-degassed and purged with N₂ for 3 times), and then aqueous solution of CuSO₄ (0.4 M, 81.1 μL, 2.0 eq.) and VcNa (12.86 mg, 64.92 μmol, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 3 was consumed completely, and one main peak with desired m/z (calculated MW: 5161.94, observed m/z: 1291.6 ([M/4+H]⁺), 1033.4 ([M/5+H]⁺), 861.4 ([M/6+H]⁺)) was detected. The reaction mixture was filtered to remove the insoluble residue. The crude product was purified by prep-HPLC (TFA condition), and Compound 5 (BCY17230, 65.0 mg, 12.1 μmol, 37.28% yield, 96.1% purity) was obtained as a white solid.

Preparation of Intermediate BCY17233

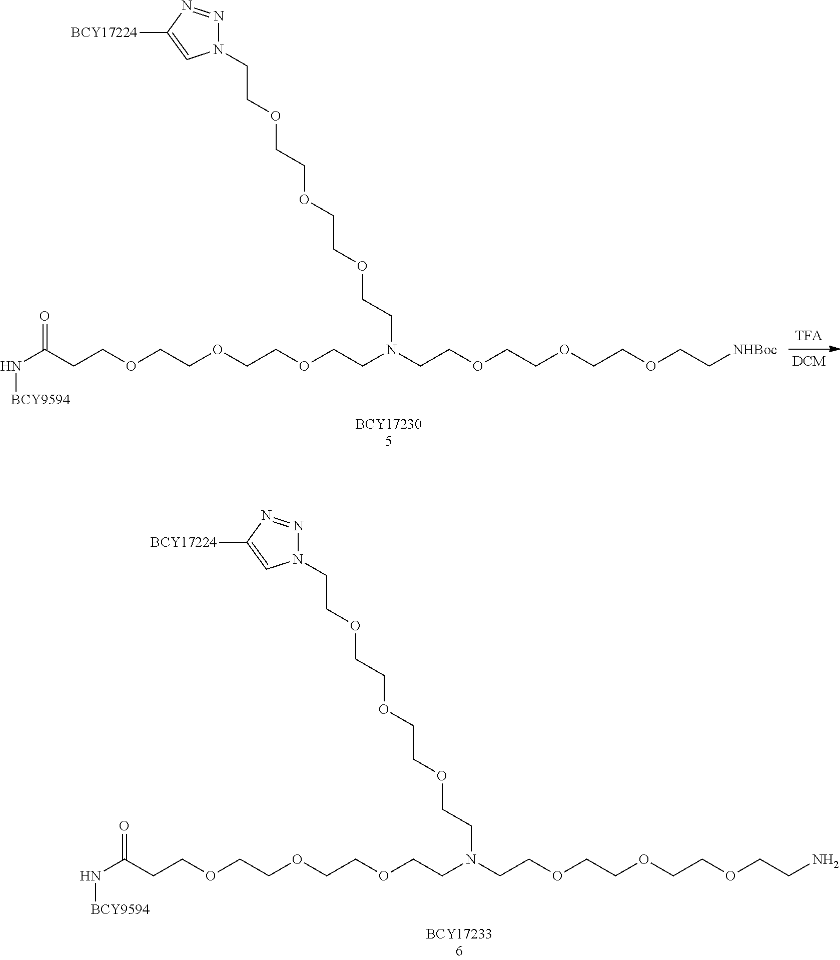

To a solution of Compound 5 (65.0 mg, 12.1 μmol, 1.0 eq) in DCM (0.80 mL) was added TEA (0.2 mL). The mixture was stirred at 25° C. for 0.5 hr. LC-MS showed compound 5 was consumed completely and one main peak with desired m/z (MW: 5061.9, observed m/z: 1266.1[M/4+H]⁺, 1013.2[M/5+H]⁺, 844.5 [M/6+H]⁺) was detected. The reaction mixture was filtered, and soluble fraction was then purified by prep-HPLC (TFA condition). Compound 6 (36.8 mg, 7.13 μmol, 56.64% yield, 98.1% purity) was obtained as a white solid.

Preparation of Intermediate BCY17234

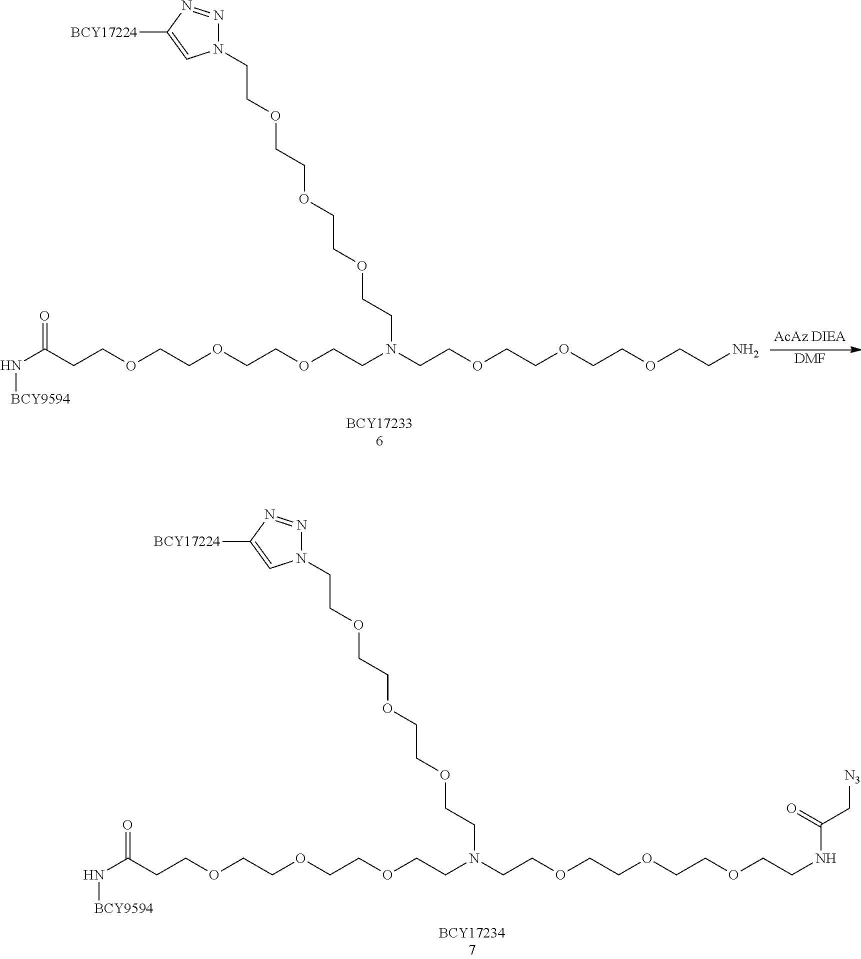

To a solution of Compound 6 (BCY17233, 36.8 mg, 7.13 μmol, 1.0 eq) and AcAz (1.73 mg, 8.72 μmol, 1.2 eq) in DMF (1.0 mL) was added DIEA (1.9 mg, 14.4 μmol, 2.5 UL, 2.0 eq). The mixture was stirred at 25° C. for 0.5 hrs. LC-MS showed compound 6 was consumed completely and one main peak with desired m/z (MW: 5141.35, observed m/z: 1029.8[M/5+H]⁺, 1286.9[M/4+H]⁺) was detected. The reaction mixture was filtered, and soluble fraction was then purified by prep-HPLC (TFA condition). Compound 7 (BCY17231, 25.0 mg, 4.65 μmol, 63.96% yield, 95.7% purity) was obtained as a white solid.

Preparation of BCY17231

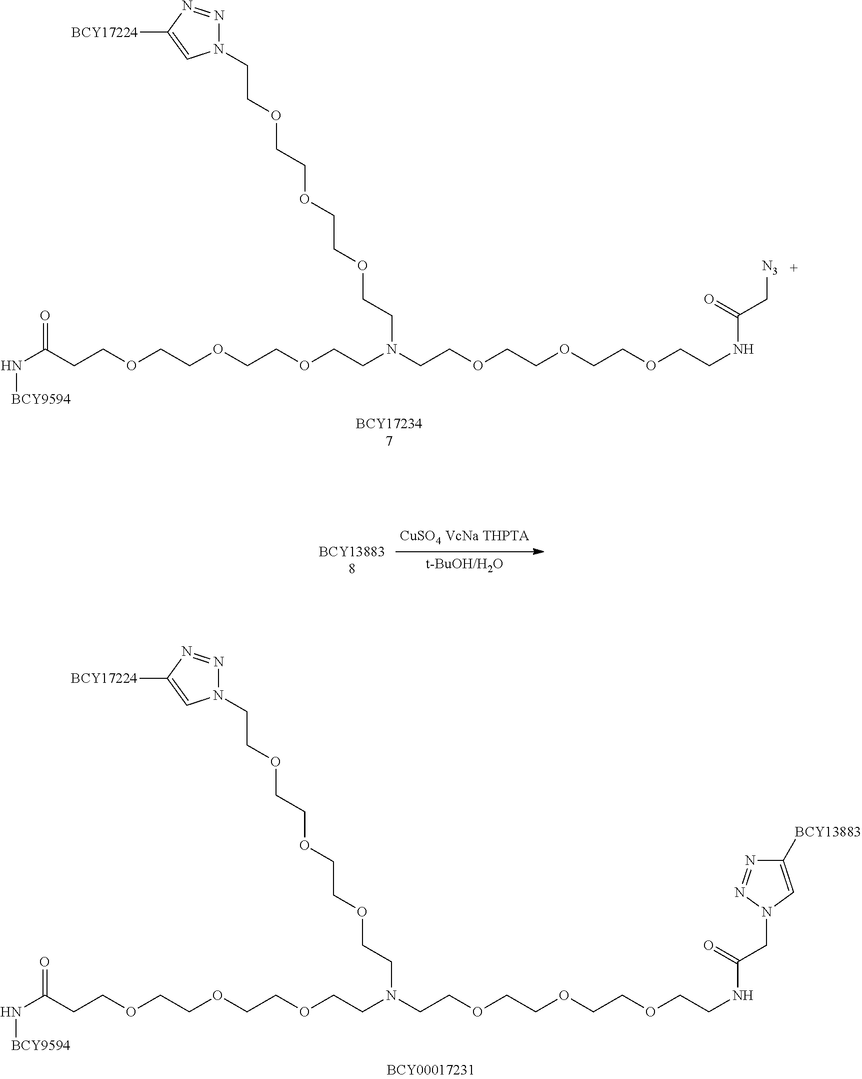

A mixture of compound 7 (22 mg, 4.28 μmol, 1.0 eq.), BCY13883 (9.31 mg, 4.70 μmol, 1.1 eq), and THPTA (1.86 mg, 4.28 μmol, 1.0 eq) was dissolved in t-BuOH/H₂O (1:1, 2.0 mL, pre-degassed and purged with N₂ for 3 times), and then aqueous solution of CuSO₄ (0.4 M, 10.69 μL, 1.0 eq) and VcNa (1.69 mg, 8.55 μmol, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 7 was consumed completely, and one main peak with desired m/z (calculated MW: 7123.1, observed m/z: 1425.3 ([M/5+H]⁺), 1188.0 ([M/6+H]⁺)) was detected. The reaction mixture was filtered to remove the insoluble residue. The crude product was purified by prep-HPLC (TFA condition), and BCY17231 (4.8 mg, 6.29e-1 μmol, 14.70% yield, 93.2% purity) was obtained as a white solid.

### Example 16: Synthesis of BCY20793

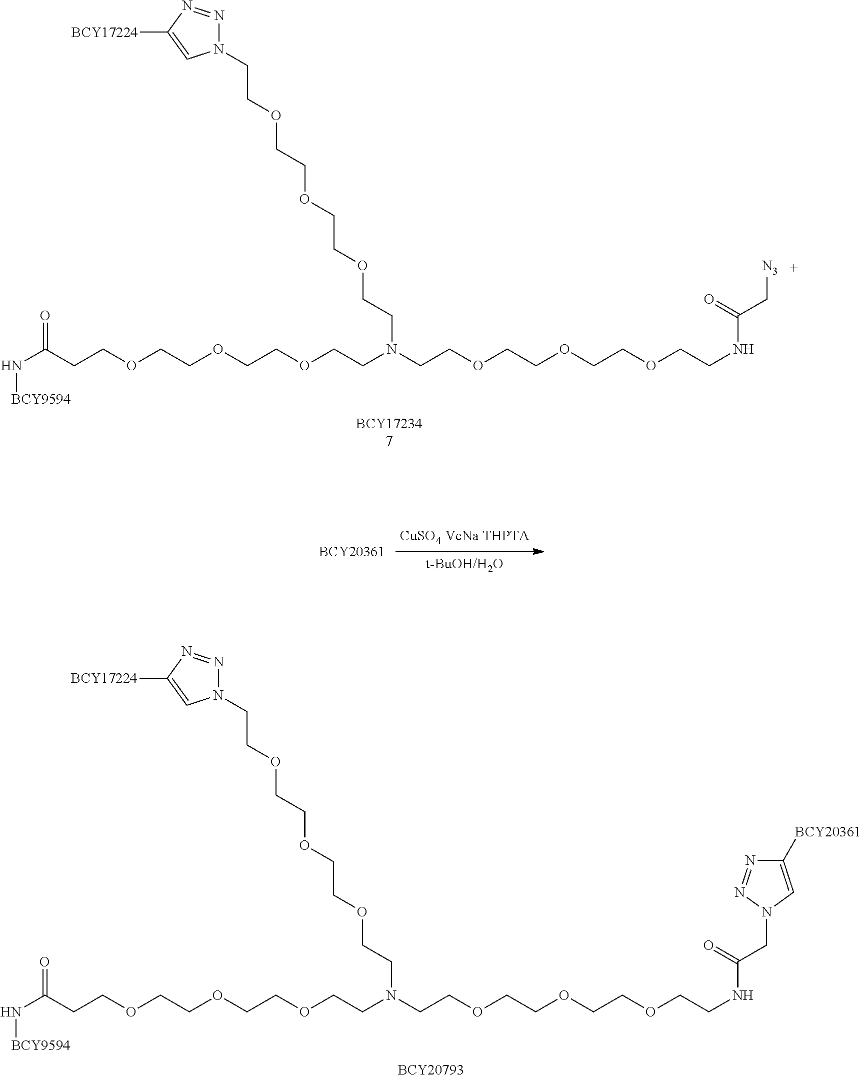

A mixture of compound 1 (17.4 mg, 3.38 μmol, 1.0 eq), BCY20361 (7.61 mg, 3.72 μmol, 1.1 eq), and THPTA (1.47 mg, 3.38 μmol, 1.0 eq) was dissolved in t-BuOH/H₂O (1:1, 2.0 mL, pre-degassed and purged with N₂ for 3 times), and then aqueous solution of CuSO₄ (0.4 M, 8.46 μL, 1.0 eq) and VcNa (1.34 mg, 6.76 μmol, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 1 was consumed completely, and one main peak with desired m/z (calculated MW: 7193.2, observed m/z: 1439.3 ([M/5+H]⁺), 1199.7 ([M/6+H]⁺)) was detected. The reaction mixture was filtered to remove the insoluble residue. The crude product was purified by prep-HPLC (TFA condition), and BCY20793 (8.0 mg, 1.05 μmol, 30.96% yield, 94.1% purity) was obtained as a white solid.

### Example 17: Synthesis of BCY17235

Preparation of Intermediate COM00000116-BCY17224

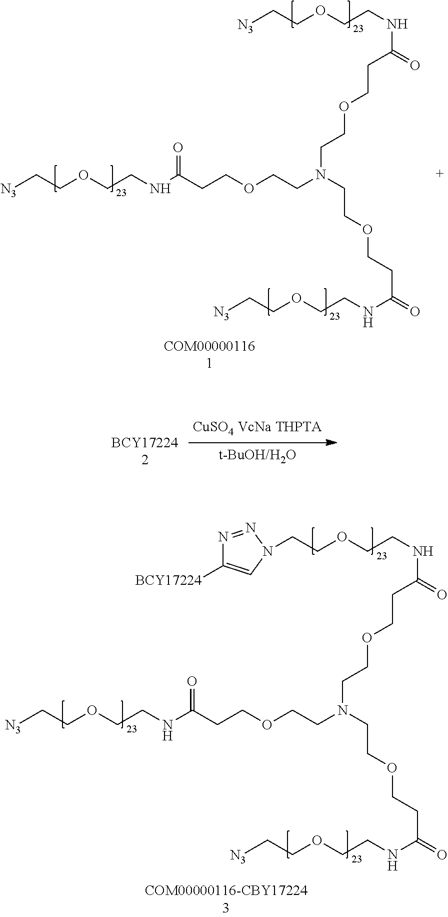

A mixture of COM00000116 (50.0 mg, 13.8 μmol, 1.0 eq.), BCY17224 (23.0 mg, 11.1 μmol, 0.8 eq.), and THPTA (30.1 mg, 69.3 μmol, 5.0 eq.) was dissolved in t-BuOH/H₂O (1:1, 10 mL, pre-degassed and purged with N₂ for 3 times), and then aqueous solution of CuSO₄ (0.4 M, 13.8 μL, 0.4 eq.) and VcNa (54.8 mg, 277.1 μmol, 20.0 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 3 hr under N₂ atmosphere. LC-MS showed desired mass (calculated MW: 5690.6, observed m/z: 1416.3 [(M-28)/4+H]⁺). The crude product was purified by prep-HPLC (TFA condition), but the purity is poor. Compound 3 (COM00000116-BCY17224, 43.9 mg, 53.1% purity) was obtained as a white solid.

Preparation of Intermediate COM00000116-BCY17224-BCY15017

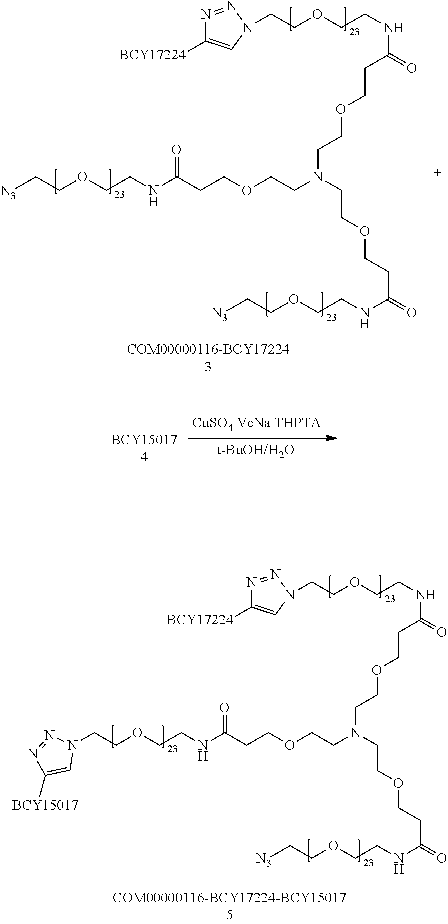

A mixture of compound 3 (COM00000116-BCY17224, 29.8 mg, 5.24 μmol, 1.0 eq.), compound 4 (BCY15017, 13.0 mg, 5.24 μmol, 1.0 eq.), and THPTA (4.5 mg, 10.5 μmol, 2.0 eq.) was dissolved in t-BuOH/H₂O (1:1, 1 mL, pre-degassed and purged with N₂ for 3 times), and then aqueous solution of CuSO₄ (0.4 M, 13.1 μL, 1.0 eq.) and VcNa (3.1 mg, 15.7 μmol, 3.0 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed desired m/z (calculated MW: 8171.45, observed m/z: 1362.6 ([M/6+H]⁺), 1168.3 ([M/7+H]⁺)) was detected. The reaction mixture was filtered to remove the insoluble residue. The soluble crude product was purified by prep-HPLC (TFA condition), and compound 5 (COM00000116-BCY17224-BCY15017, 8.7 mg, 1.06 μmol, 20.2% yield, 78.7% purity) was obtained as a white solid.

Preparation of BCY17235

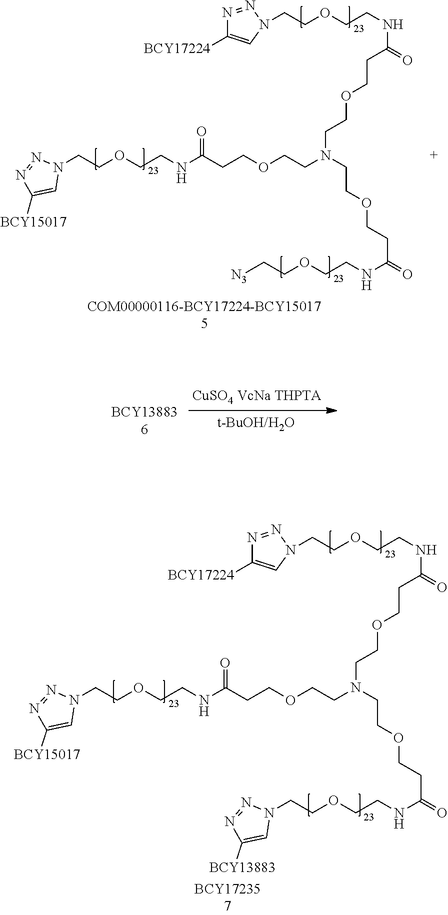

A mixture of compound 5 (COM00000116-BCY17224-BCY15017, 4.2 mg, 5.14e-1 μmol, 1.0 eq.), compound 6 (BCY13883, 1.0 mg, 5.14e-1 μmol, 1.0 eq.), and THPTA (0.2 mg, 5.14e-1 μmol, 1.0 eq.) was dissolved in t-BuOH/H₂O (1:1, 0.2 mL, pre-degassed and purged with N₂ for 3 times), and then aqueous solution of CuSO₄ (0.4 M, 1.3 μL, 1.0 eq.) and VcNa (0.2 mg, 1.03 μmol, 2.0 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 0.5 hr under N₂ atmosphere. LC-MS showed compound 5 was consumed completely, and one main peak with desired m/z (calculated MW: 10149.8, observed m/z: 1269.3 ([M/8+H]⁺), 1128.4 ([M/9+H]⁺)) was detected. The reaction mixture was filtered to remove the insoluble residue. The soluble crude product was purified by prep-HPLC (TFA condition), and BCY17235 (1.8 mg, 1.77e-1 μmol, 34.4% yield, 91.6% purity) was obtained as a white solid.

### Example 18: Synthesis of BCY17225

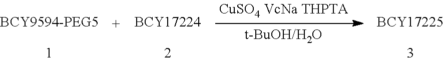

A mixture of compound 1 (20 mg, 7.36 μmol, 1.0 eq), compound 2 (BCY17224, 16.85 mg, 8.09 μmol, 1.1 eq), and THPTA (3.20 mg, 7.36 μmol, 1.0 eq) was dissolved in t-BuOH/H₂O (1:1, 1.0 mL, pre-degassed and purged with N₂ for 3 times), and then aqueous solution of CuSO₄ (0.4 M, 18.4 μL, 1.0 eq) and VcNa (2.92 mg, 14.72 μmol, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 1 was consumed completely, and one main peak with desired m/z (calculated MW: 4799.47, observed m/z: 961.0 ([M/5+H]⁺), 1200.9 ([M/4+H]⁺)) was detected. The reaction mixture was filtered to remove the insoluble residue. The crude product was purified by prep-HPLC (TFA condition), and BCY17225 (19.5 mg, 3.88 μmol, 52.73% yield, 95.5% purity) was obtained as a white solid.

### Example 19: Synthesis of BCY18731

Preparation of Intermediate N3-PEG₂₄-BCY9594

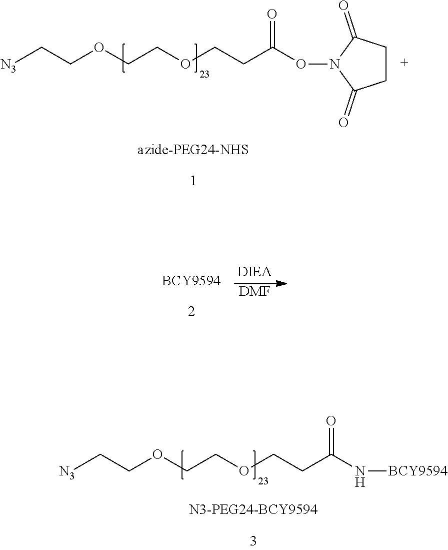

A mixture of compound 1 (17.45 mg, 13.75 μmol, 1.1 eq), DIEA (3.23 mg, 24.99 μmol, 4.35 uL, 2.0 eq) was dissolved in DMF (1.0 mL). The reaction mixture was activated at 25-30° C. for 6 min, then compound 2 (30.0 mg, 12.50 μmol, 1.0 eq) was added into the reaction mixture. The reaction mixture was stirred at 25-30° C. for 0.5 hr. LC-MS showed compound 2 was consumed completely and one main peak with desired m/z (MW: 3555.1, observed m/z: 1186.1 [(M/3+H]⁺) was detected. The reaction mixture was filtered to remove the insoluble residue. The residue was then purified by prep-HPLC (TFA condition). Compound 3 (N3-PEG24-BCY9594, 23.8 mg, 6.69 μmol, 52.40% yield, 97.8% purity) was obtained as a white solid.

Prepraration of BCY 18731

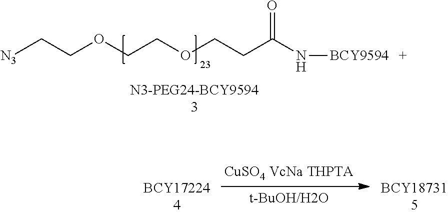

A mixture of compound 3 (N3-PEG24-BCY9594, 23.8 mg, 6.69 μmol, 1.0 eq), compound 4 (BCY17224, 15.33 mg, 7.36 μmol, 1.1 eq) and THPTA (2.91 mg, 6.69 μmol, 1.0 eq) was dissolved in t-BuOH/H₂O (1:1, 2.0 mL, pre-degassed and purged with N₂ for 3 times), and then aqueous solution of CuSO₄ (0.4 M, 16.74 L, 1.0 eq) and VcNa (2.65 mg, 13.39 μmol, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 1 hr under N₂ atmosphere. LC-MS showed compound 3 was consumed completely, and one main peak with desired m/z (calculated MW: 5636.47, observed m/z: 1410.0 ([M/5+H]⁺), 1128.3 ([M/5+H]⁺), 940.4 ([M/6+H]⁺)) was detected. The reaction mixture was filtered to remove the insoluble residue. The crude product was purified by prep-HPLC (TFA condition), and BCY18731 (14.7 mg, 2.50 μmol, 37.4% yield, 96.0% purity) was obtained as a white solid.

### BIOLOGICAL DATA

### 1. Degranulation Assay

Tumor cells can be recognized and killed by CD8+T-cytotoxic and NK cells through the immune secretion of lytic granules that kill target cells. This process involves the fusion of the granule membrane with the cytoplasmic membrane of the immune effector cell, resulting in surface exposure of CD107a (LAMP1). Membrane expression of CD107a constitutes a marker of immune cell activation and cytotoxic degranulation. EphA2/NKp46 or EphA2/CD16a heterotandem bicyclic peptide complexes were evaluated for activation of NK cells using a degranulation assay.

On the day of the experiment, medium was prepared by supplementing RPMI-1640 (Gibco™ 11875-093; with L-glutamine) with 10% heat-inactivated fetal bovine serum (FBS; Corning® 35-011-CV), 10 mM HEPES (Gibco™ 15-630-080), and 1% Penicillin Streptomycin (Corning™ 30-002-CI), herein referred to as working medium. Previously isolated human peripheral blood mononuclear cells (PBMCs), from whole blood, were quick thawed in a water bath and washed once at 500 rpm for 5 minutes in 10 mL of prewarmed working medium. PBMC pellet was then resuspended to concentration of 5×10⁶ cells/mL in working medium and rested overnight (12-18 hours) horizontally in a tissue-culture coated flask (T-183; CELLTREAT Scientific 229351). Moreover, Ephrin type-A receptor (EphA2) expressing human lung carcinoma cell line A549 (ATCCR CCL-185; cells were grown and maintained according to manufacturer's recommendation) were detached from the culture vessel using Trypsin/EDTA and washed once at 500 rpm for 5 minutes in 15 mL of prewarmed working medium. Cell pellet was then resuspended in working medium at a concentration of 1×10⁵ cells/mL. Subsequently, 100 μL of cell suspension was plated in a flat-bottom tissue-culture coated 96-well plate (Greiner CellStar® 655180) and rested overnight (12-18 hours).

Post-overnight incubation, PBMCs were removed from the flask and washed once at 500 rpm for 5 minutes in 10 mL of prewarmed working medium. NK cells were subsequently isolated from the total PBMC population using a negative isolation kit (STEMCELL Technologies 17955) according to manufacturer's recommendation. NK cell pellet was then resuspended in working medium at a concentration of 1×10⁵ cells/mL. Subsequently, 100 μL of cell suspension was plated in the 96-well plate containing the A549 overnight rested cells.

Heterotandem bicyclic peptide complexes were diluted in working medium and added to the corresponding cell plate at a suggested starting concentration of 300 nM or 5 UM titrated in a 1:4 dilution series to perform a 12-point serial dilution. Additionally, a protein transport inhibitor, GolgiStop™ (BD Biosciences 554715), was added according to manufacturer's recommendation. Plates were then incubated for 4 hours at 37° C., 5% CO₂. Samples were then washed once in 200 μL of 1× phosphate buffer saline (PBS; Gibco™ 10-010-023) at 500 rpm for 5 minutes. Cells were resuspended in 200 μL of PBS and transferred to a 96-well V-bottom polypropylene plate (Greiner Bio-One 651201). Samples were then centrifuged at 500 rpm for 5 minutes and supernatant was discarded.

Preparation of samples for flow cytometry Zombie Aqua™ Fixable Viability Dye (BioLegend® 423102) was prepared as a 1:1000 dilution in PBS and 100 μL of viability dye was added to each well and incubated in the dark at 4° C. for 30 minutes. Subsequently, wells were washed with 100 μL of PBS for 5 minutes at 500 rpm and supernatant was discarded. Next, human TruStain FcX™ block (BioLegend® 422302) was prepared by diluting 1.5 μL of FcX in 25 μL of stain buffer (1×PBS supplemented with 2% FBS). Fc block solution (25 μL/well) was incubated at room temperature (RT) for 10 minutes in the dark. Antibody master mix cocktail was prepared by diluting 1.5 L the following antibodies per 100 μL of stain buffer: FITC anti-human CD45 (BioLegend® 304038; clone HI30), Brilliant Violet 605™ anti-human CD3 (BioLegend® 344836; clone SK7), PE/Cyanine7 anti-human CD56 (BioLegend® 362510; clone 5.1H11), PE anti-human NKp46 (BioLegend® 331908; clone 9E2), and Brilliant Violet 421 ™ anti-human CD107a (BioLegend® 328626; clone H4A3). Cells were resuspended in master mix cocktail (100 μL) and incubated at 4° C. for 30 minutes in the dark. Subsequently, cells were washed 3 times in 100 μL of stain buffer for 5 minutes at 500 rpm and supernatant was discarded. Cells resuspended in 200 μL of stain buffer were kept at 4° C. and in the dark until read by BD FACSCelesta™ flow cytometer and FCS files were analyzed in FlowJo™.

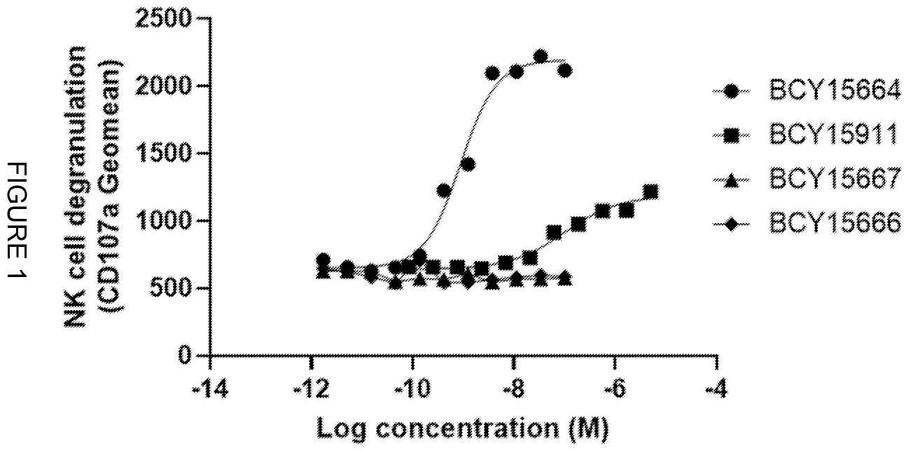

The data shown in Table 1 and FIG. 1 illustrate dose-dependent upregulation of CD107a on NK cell surface post-treatment with BCY15664 and BCY15911. Non-binding controls, BCY15667 and BCY15666, elicit no upregulation above baseline.

The data shown in Table 1 and FIG. 1 illustrate dose-dependent and tumor-antigen dependent upregulation of CD107a on NK cell surface post-treatment with BCY 15664 and BCY15911. Non-binding control (BCY15667) with both the EphA2 and NKp46 bicyclic peptides comprised of all D-amino acids elicits no upregulation above baseline. Additionally, control (BCY15666) with only the EphA2 bicyclic peptide comprised of all D-amino acids and which therefore is competent for binding to NKp46 but not EphA2, elicits no upregulation above baseline.

TABLE 1
Half maximal effective concentration (EC₅₀)
associated with the CD107a surface expression
on NK cells indicating degranulation

| Heterotandem | EC50 Degranulation |
|---|---|
| Complex Number | Geometric Mean CD107a (nM) |
| BCY15664 | 0.88 |
| BCY15911 | 99.82 |
| BCY15667 | N/A |
| BCY15666 | N/A |

### 2. NK Cytotoxicity Assay

Heterotandem bicyclic peptide complexes were evaluated for NK functional readouts (cytotoxicity and cytokine secretion) in NK-tumor cell line co-cultures.

NK cells were isolated using a negative isolation kit (STEMCELL™ Technologies 17955) from the total PBMC population purified from whole blood. NK cell pellet was then resuspended in DMEM medium (Corning™ 10-013-CV) with 10% heat-inactivated fetal bovine serum (FBS; Corning® 35-011-CV), 10 mM HEPES (Gibco™ 15-630-080), and 1% Penicillin Streptomycin (Corning™ 30-002-CI), and 50 IU/mL human IL-2 (Miltenyi Biotec® 130-097-748), at a concentration of 4×10⁵ cells/mL. For the NK cell cytotoxicity assays, 50 μL (2×10⁴) of cell suspension was plated in the 96-well plate (Grenier® Bio One™ 655090) containing 50 μl (4×10³) HT1080-luc cells (ATCC® CCL-121-luc2) in DMEM (Gibco™ 11875-093; with L-glutamine) with 10% heat-inactivated fetal bovine serum (FBS; Corning® 35-011-CV), 10 mM HEPES (Gibco™ 15-630-080), and 1% Penicillin Streptomycin (Corning™ 30-002-CI). Test articles or antibody (InvivoGen® hegfr-mab1) were diluted in DMEM medium (Corning™ 10-013-CV) with 10% heat-inactivated fetal bovine serum (FBS; Corning® 35-011-CV), 10 mM HEPES (Gibco™ 15-630-080), and 1% Penicillin Streptomycin (Corning™ 30-002-CI)) and added to the corresponding cell plate (50 μl) at a suggested starting concentration of 10 nM titrated in a ⅕ dilution series to perform an 8-point serial dilution. The plates were then incubated for 24 hours at 37° C., 5% CO₂. Post-incubation, plates was centrifuged at 250×g for 5 minutes and 100 μl supernatant discarded. Samples were then incubated 10 minutes with 50 μl Bright-Glo™ Luciferase Assay system (Promega™ E2620). Luminescence upon excitation at 570 nm was read using a CLARIOstar® plate-reader with MARS Data Analysis Software™. Data was fit to a four-parameter non-linear regression in GraphPad Prism™ 8.0.2 to generate an EC50 value.

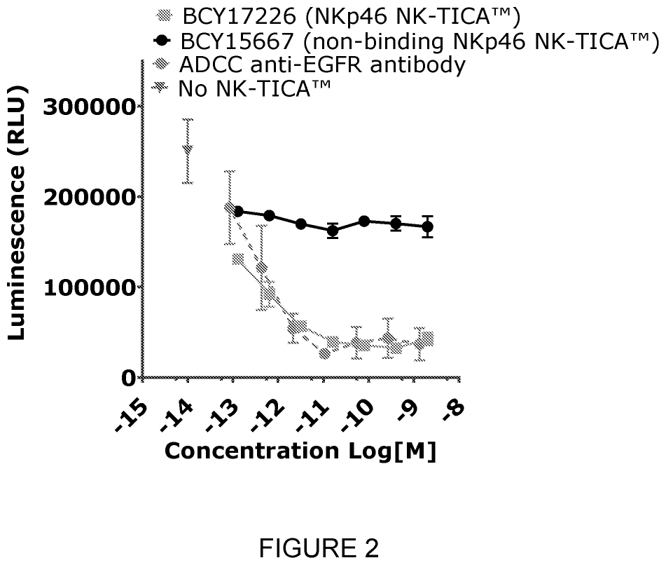

Selected heterotandem bicyclic peptide complexes of the invention were tested in this assay and the results are shown in FIGS. 2 to 4 and 6 to 10.

FIG. 2 illustrates that BCY17226 elicits a dose-dependent NK cell response to kill EphA2+ve HT1080-luc tumor cell line. No enhanced, dose-dependent effect in tumor cell killing is observed with non-binding heterotandem bicyclic peptide complex (BCY15667) in comparison to NK:HT 1080-luc coculture without addition of a heterotandem bicyclic peptide complex. An ADCC-capable anti-EGFR antibody (InvivoGen®, hegfr-mab1) was used in the assay as a positive reference control for NK-induced cytotoxicity. Average luminescence for No NK-TICA (referring to “No Heterotandem bicyclic peptide complex”) is arbitrarily shown at 0.00001 pM for reference. The EC₅₀=6.1 pM of BCY17226 was calculated using a four-parameter logistic regression using GraphPad Prism™ 8.0.2.

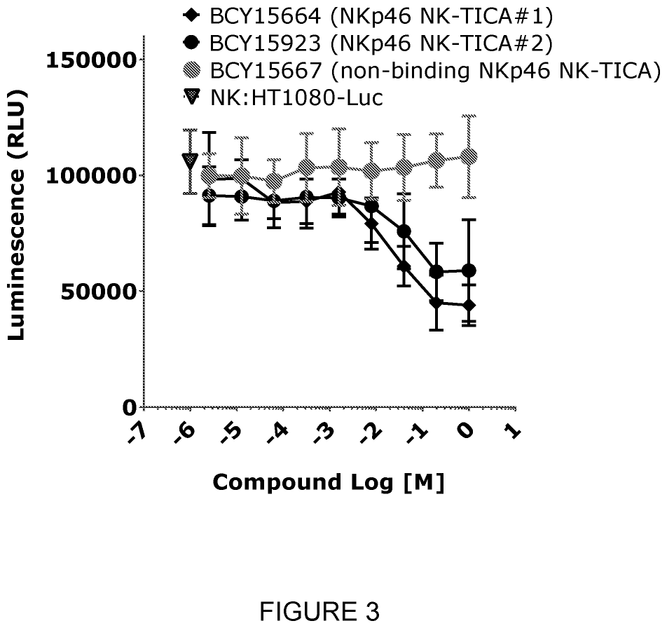

FIG. 3 illustrates that BCY15664 and BCY15923 elicit a dose-dependent NK cell response to kill EphA2+ve HT1080-luc tumor cell line. No enhanced, dose-dependent effect in tumor cell killing is observed with non-binding heterotandem bicyclic peptide complex (BCY15667) in comparison to NK:HT1080-luc coculture without addition of a heterotandem bicyclic peptide complex. An ADCC-capable anti-EGFR antibody (InvivoGen®, hegfr-mab1) was used in the assay as a positive reference control for NK-induced cytotoxicity. Average luminescence for No NK-TICA (referring to “No heterotandem bicyclic peptide complex”) is arbitrarily shown at 0.00001 pM for reference. BCY15664 (NKp46 epitope 1, EC₅₀=21 pM) or BCY15923 (NKp46 epitope 2, EC₅₀=44 pM) was calculated using a four-parameter logistic regression using GraphPad Prism™ 8.0.2 software.

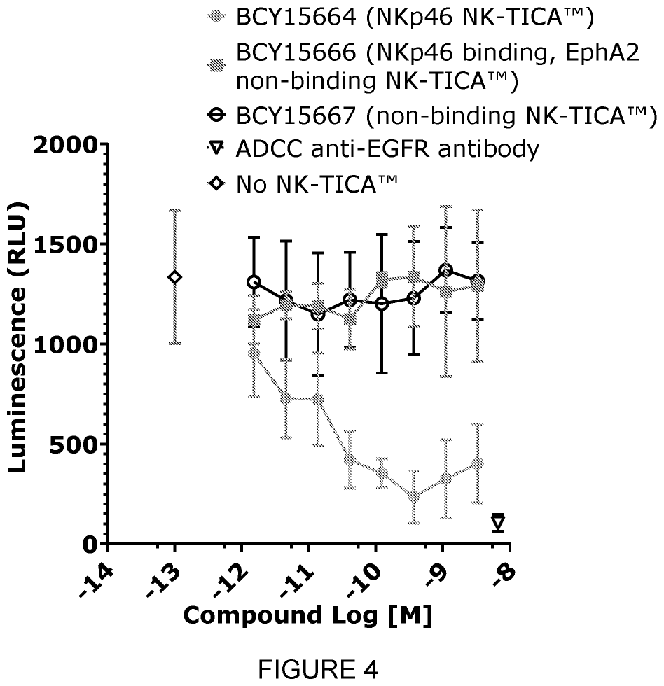

FIG. 4 illustrates the necessity of tumor antigen binding bicyclic peptides in the heterotandem bicyclic peptide complex construct to elicit enhanced NK cytotoxic activity. No enhanced, dose-dependent effect in tumor cell killing is observed with non-binding EphA2/non-binding NKp46 heterotandem bicyclic peptide complex (BCY15667) in comparison to NK:HT 1080-luc co-culture without addition of heterotandem bicyclic peptide complex. No enhanced, dose-dependent effect in tumor cell killing is observed with non-binding EphA2/binding NKp46 heterotandem bicyclic peptide complex (BCY15666) in comparison to NK:HT 1080-luc co-culture without addition of a heterotandem bicyclic peptide complex. An ADCC-capable anti-EGFR antibody (InvivoGen®, hegfr-mab1) was used in the assay as a positive reference control for NK-induced cytotoxicity. Average luminescence for No NK-TICA (referring to “No Heterotandem bicyclic peptide complex”) is arbitrarily shown at 0.001 nM for reference. The EC₅₀=16 pM of BCY15664 was calculated using a four-parameter logistic regression using GraphPad Prism™ 8.0.2.

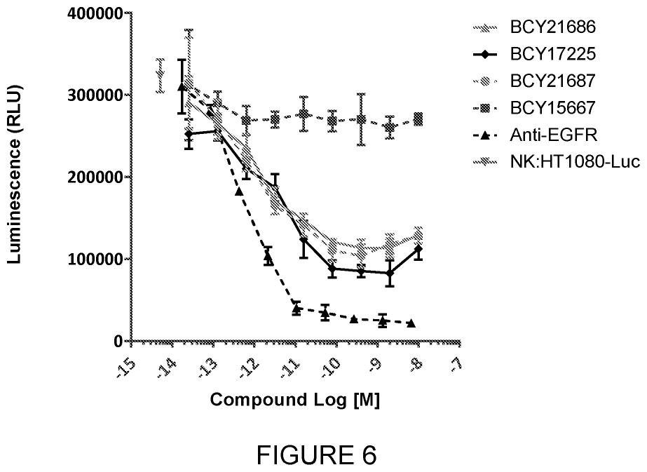

FIG. 6 illustrates enhanced NK cytotoxic activity with varying valency of Bicycle NKp46 in the NK-TICA construct. An enhanced, dose-dependent effect in tumor cell killing is observed with NK-TICA constructs: BCY17225_01_02 (EC₅₀=3.6 pM), BCY21686_01_01 (EC₅₀=1.3 pM) in comparison to NK:HT1080-luc co-culture without NK-TICA addition. No enhanced, dose-dependent effect in tumor cell killing is observed with non-binding NK-TICA (BCY15667_01_01). An ADCC-capable anti-EGFR antibody (InvivoGen®, hegfr-mab1) was used in the assay as a positive reference control for NK-induced cytotoxicity (EC₅₀=0.053 pM). Average luminescence for no NK-TICA is arbitrarily shown at 0.005 pM for reference. The EC₅₀ values of the Bicycles was calculated using a four-parameter logistic regression using GraphPad Prism™ 8.0.2.

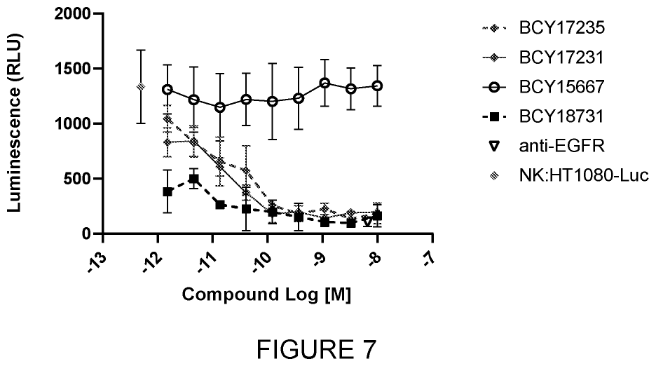

FIG. 7 illustrates enhanced dose-dependent NK cytotoxic activity with varying NK-TICA construct spacer length. An enhanced, dose-dependent effect on tumor cell killing is observed with NKp46 Bicycle NK-TICA constructs: BCY18731_01_01 (EC₅₀=18 pM), BCY17231_01_01 (EC₅₀=21 pM) and BCY17235_04_01 (EC₅₀=14 pM) in comparison to NK:HT1080-luc co-culture without NK-TICA addition. No enhanced effect on NK cytotoxicity was observed with non-binding NK-TICA (BCY15667_01_01). An ADCC-capable anti-EGFR antibody (InvivoGen®, hegfr-mab1, 6.7 nM) was used in the assay as a positive reference control for NK-induced cytotoxicity. Average luminescence for no NK-TICA is arbitrarily shown at 0.05 pM for reference. The EC₅₀ values of the Bicycles was calculated using a four-parameter logistic regression using GraphPad Prism™ 8.0.2.

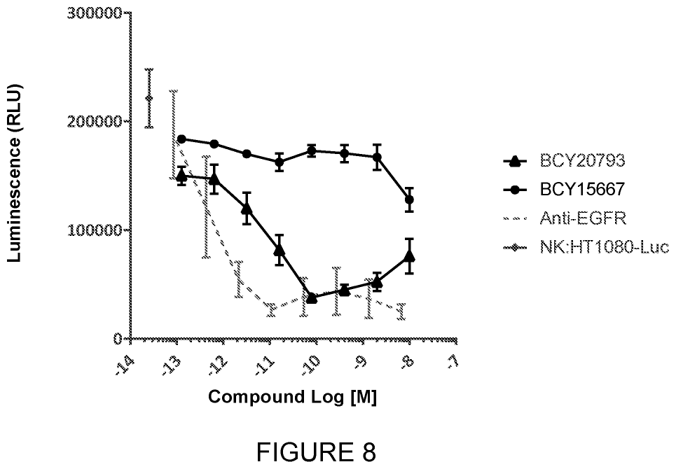

FIG. 8 illustrates enhanced dose dependent tumor killing by NK cells treated with CD16 binding Bicycle and NKp46 binding Bicycle NK-TICA construct (BCY20793_01_01). Tumoricidal activity of NK cells was observed with BCY20793_01_01 (EC₅₀=6.5 pM). An ADCC-capable anti-EGFR antibody (InvivoGen®, hegfr-mab1) used in the assay as a positive reference control for NK-induced cytotoxicity (EC₅₀=0.45 pM). No enhanced, dose-dependent effect in tumor cell killing is observed with non-binding NK-TICA (BCY15667_01_01) in comparison to NK:HT1080-luc co-culture without NK-TICA addition. Average luminescence for no NK-TICA is arbitrarily shown at 0.02 pM for reference. The EC₅₀ values of the Bicycles were calculated using a four-parameter logistic regression using GraphPad Prism™ 8.0.2.

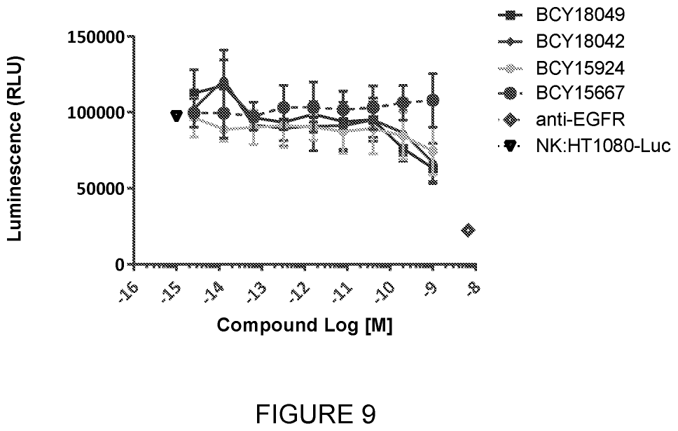

FIG. 9 illustrates the alternative NKp46 binding Bicycles in NK-TICA induce enhanced killing of HT1080-luc cells. NKp46 binding NK-TICA constructs BCY18049_01_01 (EC₅₀ =unstable), BCY18042_01_01 (EC₅₀=unstable), and BCY15924_01_01 (EC₅₀=0.5 nM), enhanced NK cytotoxicity of HT1080-luc tumor cell line in comparison to the NK:HT1080-luc co-culture without NK-TICA addition. BCY15667_01_01 (non-binding NK-TICA) does not have activity in comparison to NK:HT 1080-luc co-culture without NK-TICA addition. Average luminescence for no NK-TICA is arbitrarily shown at 0.001 pM for reference. An ADCC-capable anti-EGFR antibody (InvivoGen®, hegfr-mab1, 6.7 nM) was used in the assay as a positive reference control for NK-induced cytotoxicity. The EC₅₀ values were calculated using a four-parameter logistic regression using GraphPad Prism™ 8.0.2.

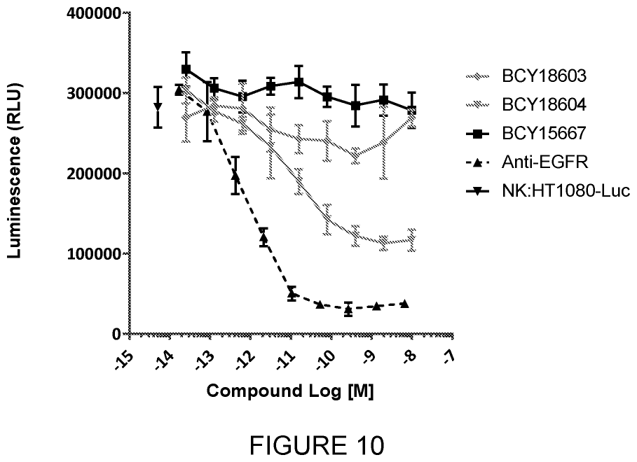

FIG. 10 illustrates NK-TICAs that include additional tumor binding Bicycle arms in the NKp46 binding NK-TICA construct induce enhanced killing of HT1080-luc cells. PD-L1 binding Bicycle NK-TICA construct (BCY18603_01_01; EC₅₀=3.0 pM), or MT1 binding Bicycle NK-TICA construct (BCY18604_01_01; EC₅₀=7.9 pM) enhanced NK cytotoxicity of HT 1080-luc tumor cell line in comparison to the NK:HT1080-luc co-culture without NK-TICA addition. The nonbinding control NK-TICA construct (BCY15667_01_02) does not have activity in comparison to NK:HT1080-luc co-culture without NK-TICA addition. Average luminescence for No NK-TICA is arbitrarily shown at 0.005 pM for reference. An ADCC-capable anti-EGFR antibody (InvivoGen®, hegfr-mab1) was used in the assay as a positive reference control for NK-induced cytotoxicity (EC₅₀=0.70 pM). EC₅₀ values were calculated using a four-parameter logistic regression using GraphPad Prism™ 8.0.2.

### 3. Cytokine Secretion Assay

Heterotandem bicyclic peptide complexes were evaluated for NK functional readouts (cytotoxicity and cytokine secretion) in NK-tumor cell line co-cultures.

NK cells were isolated using a negative isolation kit (STEMCELL Technologies® 17955) from the total PBMC population purified from whole blood. NK cell pellet was then resuspended in DMEM medium (Corning™ 10-013-CV) with 10% heat-inactivated fetal bovine serum (FBS; Corning® 35-011-CV), 10 mM HEPES (Gibco™ 15-630-080), and 1% Penicillin Streptomycin (Corning™ 30-002-CI), and 50 IU/mL human IL-2 (Miltenyi Biotec® 130-097-748), at a concentration of 4×10⁵ cells/mL. For the NK cell cytokine secretion assay, 2×10⁵ NK cell in 50 μl cell suspension were plated in the 96-well U-bottom plate (Grenier Bio One™ 650180) containing 50 μl (4×10⁴) HT1080-luc cells (ATCC® CCL-121-luc2) in DMEM (Gibco™ 11875-093; with L-glutamine) with 10% heat-inactivated fetal bovine serum (FBS; Corning® 35-011-CV), 10 mM HEPES (Gibco™ 15-630-080), and 1% Penicillin Streptomycin (Corning™ 30-002-CI). Test articles or antibody (InvivoGen®, hegfr-mab1) were diluted in DMEM medium (Corning™ 10-013-CV) with 10% heat-inactivated fetal bovine serum (FBS; Corning® 35-011-CV), 10 mM HEPES (Gibco™ 15-630-080), and 1% Penicillin Streptomycin (Corning™ 30-002-CI) and added to the corresponding cell plate (50 μl) at a suggested starting concentration of 10 nM titrated in a ⅕ dilution series to perform an 8-point serial dilution. The plates were then incubated for 4 hours at 37° C., 5% CO₂. Post-incubation, plates was centrifuged at 250×g for 5 minutes and 100 μl supernatant harvested. Samples were immediately stored at −20° C. or assessed for cytokine levels. Interferon-gamma or TNF-alpha levels were measured in 50 μl of collected supernatants through the Human IFN-gamma Quantikine™ ELISA kit (R&D Systems DIF50C) or Human TNF-alpha Quantikine™ ELISA kit (R&D Systems DTAOOD). Data was acquired at wavelength of 450 nm on CLARIOstar® plate-reader and was fit to a four-parameter non-linear regression in CLARIOstar® plate reader MARS Data Analysis™ software and analyzed with GraphPad Prism™ 8.0.2 software to quantify to cytokine levels. Alternatively, TNF-alpha and IFN-gamma cytokine were measured from 25 μl of collected supernatant by Luminex Assay: Human XL Cytokine Discovery Premixed Kit (R&D Systems, FCS™ 18-05) Luminex 200™ flow instrument and xPONENT™ analysis software.

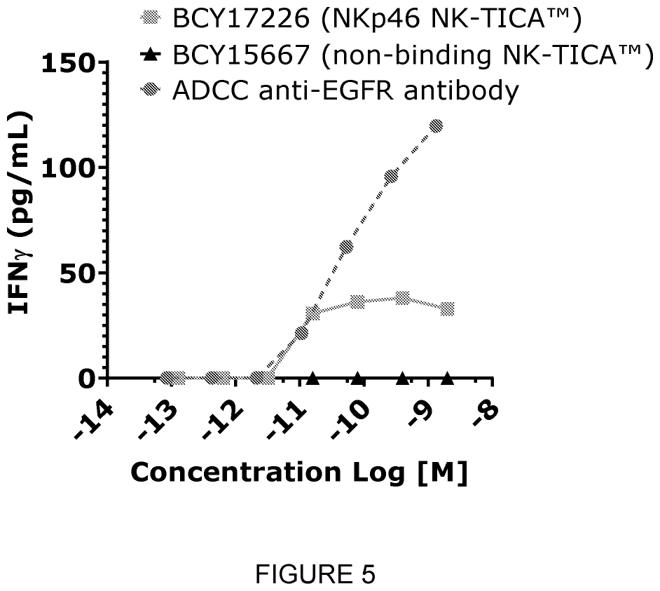

Selected heterotandem bicyclic peptide complexes of the invention were tested in this assay and the results are shown in FIGS. 5, 11 and 12.

FIG. 5 demonstrates that NK cells co-cultured with the HT 1080-luc tumor cell line in the presence of BCY17226, or non-binding heterotandem bicyclic peptide complex BCY15667. As a positive control, ADCC-capable anti-EGFR antibody (InvivoGen, hegfr-mab1) was utilized. Cytokine released (IFNγ) was measured by ELISA (R&D systems, DIF50C), applying four-parameter non-linear regression in CLARIOstar™ plate reader and MARS Data Analysis™ software.

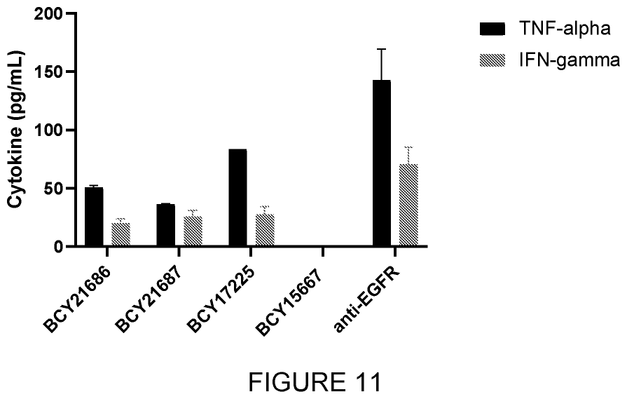

FIG. 11 illustrates NK cells produce TNF-alpha and IFN-gamma when co-cultured with the HT1080-luc tumor cell line in presence of 2 nM NKp46 binding Bicycle NK-TICA constructs of varying NKp46 Bicycle valency. Cytokine production from NK cells was observed with addition of BCY00017225_01_02, BCY21686_01_01, BCY21687_01_01 in comparison to non-binding NK-TICA construct (BCY15667_01_01). An ADCC-capable anti-EGFR antibody (InvivoGen, hegfr-mab1) was utilized as a positive reference control for NK-induced cytokine (1.34 nM). Cytokine released (IFN-gamma and TNF-alpha) was measured by Luminex Assay: Human XL Cytokine Discovery Premixed Kit (R&D Systems, FCS™18-05), by Luminex 200 flow instrument and xPONENT analysis software.

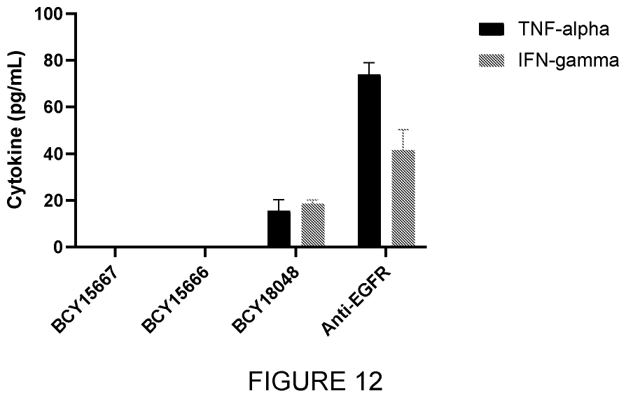

FIG. 12 illustrates NK cells secrete cytokines when co-cultured with the HT 1080-luc tumor cell line in presence of 10 nM NKp46 binding Bicycle NK-TICA construct with structural modifications. The NKp46 binding Bicycle NK-TICA (BCY18048_01_01) induced secretion of IFN-gamma and TNF-alpha compared to no cytokine production with treatment with the non-binding NK-TICA (BCY15667_01_01 or BCY15666_01_01). As a positive control, ADCC-capable anti-EGFR antibody (InvivoGen, hegfr-mab1) was utilized at 6.7 nM. Cytokine released (IFN-gamma and TNF-alpha) was measured by ELISA (R&D systems, DIF50C, DTAOOD), applying four-parameter non-linear regression in CLARIOstar plate reader and MARS Data Analysis Software.

### LIST OF REFERENCES

Chinnery F et al Oncoimmunology. 2012 Sep. 1; 1(6):874-883. doi: 10.4161/onci.20636. PMID: 23162755; PMCID: PMC3489743.
Arnon T I et al Blood. 2004 Jan. 15; 103(2):664-72. doi: 10.1182/blood-2003-05-1716. Epub 2003 Sep. 22. PMID: 14504081.
Moretta L et al Semin Immunol. 2006 June; 18(3): 151-8. doi: 10.1016/j.smim.2006.03.002. Epub 2006 May 26. PMID: 16730454.
Costello R T et al Blood. 2002 May 15; 99(10):3661-7. doi: 10.1182/blood.v99.10.3661. PMID: 11986221.
Garcia-Iglesias T et al BMC Cancer. 2009 Jun. 16; 9:186. doi: 10.1186/1471-2407-9-186. PMID: 19531227; PMCID: PMC2704222.
Nayyar G et al Front Oncol. 2019 Feb. 11; 9:51. doi: 10.3389/fonc.2019.00051. PMID: 30805309; PMCID: PMC6378304.
Stojanovic A, Cerwenka A. J Innate Immun. 2011; 3(4):355-64. doi: 10.1159/000325465. Epub 2011 Apr. 18. PMID: 21502747.
Sordo-Bahamonde C et al Cell Immunotherapy. Cancers (Basel). 2020 Apr. 7; 12(4):893. doi: 10.3390/cancers 12040893. PMID: 32272610; PMCID: PMC7226138.
Watanabe M et al Dis Esophagus. 2010 November; 23(8):675-81. doi: 10.1111/j.1442-2050.2010.01073.x. PMID: 20545975.
Izawa S et al Cancer Immunol Immunother. 2011 December; 60(12): 1801-10. doi: 10.1007/s00262-011-1082-7. Epub 2011 Aug. 3. PMID: 21811786.
Koo K C et al PLOS One. 2013 Nov. 4; 8(11): e78049. doi: 10.1371/journal.pone.0078049. PMID: 24223759; PMCID: PMC3817174.
Sun C et al Cell Mol Immunol. 2015 May; 12(3):292-302. doi: 10.1038/cmi.2014.91. Epub 2014 Oct. 13. PMID: 25308752; PMCID: PMC4654321.
Hasmim M et al Front Immunol. 2015 Sep. 23; 6:482. doi: 10.3389/fimmu.2015.00482. PMID: 26441986; PMCID: PMC4585210.
Han B et al J Immunol Res. 2018 Sep. 3; 2018:6248590. doi: 10.1155/2018/6248590. PMID: 30255106; PMCID: PMC6140275.
Stringaris K et al Haematologica. 2014 May; 99(5):836-47. doi: 10.3324/haematol.2013.087536. Epub 2014 Jan. 31. PMID: 24488563; PMCID: PMC4008119.
Rocca Y S et al Front Immunol. 2016 Oct. 10; 7:413. doi: 10.3389/fimmu.2016.00413. PMID: 27777574; PMCID: PMC5056190.
Gauthier L et al Cell. 2019 Jun. 13; 177(7):1701-1713.e16. doi: 10.1016/j.cell.2019.04.041. Epub 2019 May 30. PMID: 31155232.
Fauriat C et al Blood. 2007 Jan. 1; 109(1):323-30. doi: 10.1182/blood-2005-08-027979. Epub 2006 Aug. 29. PMID: 16940427.
Mamessier E et al Cancer Res. 2011 Nov. 1; 71(21):6621-32. doi: 10.1158/0008-5472.CAN-11-0792. Epub 2011 Sep. 21. PMID: 21937679.
Platonova S et al Cancer Res. 2011 Aug. 15; 71(16):5412-22. doi: 10.1158/0008-5472.CAN-10-4179. Epub 2011 Jun. 27. PMID: 21708957.
Levi I et al Oncotarget. 2015 May 30; 6(15): 13835-43. doi: 10.18632/oncotarget.3453. PMID: 26079948; PMCID: PMC4537053.
Kim H S et al Immunity. 2010 Feb. 26; 32(2): 175-86. doi: 10.1016/j.immuni.2010.02.004. PMID: 20189481; PMCID: PMC2843589.
MacFarlane A W 4th et al Oncoimmunology. 2017 May 19; 6(7): e1330235. doi: 10.1080/2162402X.2017.1330235. PMID: 28811973; PMCID: PMC5543845.
Muta T et al Erratum in: Nature. 1994 May 26; 369(6478):340. PMID: 8107887.
Ravetch J V, Bolland S. IgG Fc receptors. Annu Rev Immunol. 2001; 19:275-90. doi: 10.1146/annurev.immunol. 19.1.275. PMID: 11244038.
Arnould L et al Br J Cancer. 2006 Jan. 30; 94(2):259-67. doi: 10.1038/sj.bjc.6602930. PMID: 16404427; PMCID: PMC2361112.
Zhang W et al J Clin Oncol. 2007 Aug. 20; 25(24):3712-8. doi: 10.1200/JCO.2006.08.8021. PMID: 17704420.,
Zhang X et al Mol Immunol. 2020 March; 119:48-58. doi: 10.1016/j.molimm.2020.01.009. Epub 2020 Jan. 21. PMID: 31978707.,
Wu S Y et al Mol Cancer. 2020 Aug. 6; 19(1): 120. doi: 10.1186/s12943-020-01238-x. PMID: 32762681; PMCID: PMC7409673.
Manches O et al Blood. 2003 Feb. 1; 101(3):949-54. doi: 10.1182/blood-2002-02-0469. Epub 2002 Oct. 3. PMID: 12393572.
Clynes R A et al Nat Med. 2000 April; 6(4):443-6. doi: 10.1038/74704. PMID: 10742152.
Riddle D S et al Hum Gene Ther. 2005 July; 16(7):830-44. doi: 10.1089/hum.2005.16.830. PMID: 16000065.
Rataj F et al Br J Cancer. 2019 January; 120(1):79-87. doi: 10.1038/s41416-018-0341-1. Epub 2018 Nov. 15. PMID: 30429531; PMCID: PMC6325122.
Caratelli S et al Front Immunol. 2017 Apr. 27; 8:457. doi: 10.3389/fimmu.2017.00457. PMID: 28496440; PMCID: PMC5406408.
Felices M et al Methods Mol Biol. 2016; 1441:333-46. doi: 10.1007/978-1-4939-3684-7_28. PMID: 27177679; PMCID: PMC5823010.
Moore G L et al MAbs. 2011 November-December; 3(6):546-57. doi: 10.4161/mabs.3.6.18123. Epub 2011 Nov. 1. PMID: 22123055; PMCID: PMC3242841.
Gleason M K et al Blood. 2014 May 8; 123(19):3016-26. doi: 10.1182/blood-2013-10-533398. Epub 2014 Mar. 20. PMID: 24652987; PMCID: PMC4014844.
Davis Z B et al Semin Immunol. 2017 June; 31:64-75. doi: 10.1016/j.smim.2017.07.011. Epub 2017 Sep. 5. PMID: 28882429; PMCID: PMC5632228.
Felices M et al Blood Adv. 2019 Mar. 26; 3(6):897-907. doi: 10.1182/bloodadvances.2018029371. PMID: 30890546; PMCID: PMC6436008.
Gauthier L et al Cell. 2019 Jun. 13; 177(7):1701-1713. e16. doi: 10.1016/j.cell.2019.04.041. Epub 2019 May 30. PMID: 31155232.
Ben-Shmuel A et al Front Immunol. 2020 Feb. 21; 11:275. doi: 10.3389/fimmu.2020.00275. PMID: 32153582; PMCID: PMC7046808.
Tarazona R et al Cancer Immunol Immunother. 2020 May; 69(5):879-899. doi: 10.1007/s00262-020-02532-9. Epub 2020 Mar. 4. PMID: 32130453.
Davis Z B et al Semin Immunol. 2017 June; 31:64-75. doi: 10.1016/j.smim.2017.07.011. Epub 2017 Sep. 5. PMID: 28882429; PMCID: PMC5632228.
Pasero C et al Oncotarget. 2015 Jun. 10; 6(16): 14360-73. doi: 10.18632/oncotarget.3965. PMID: 25961317; PMCID: PMC4546472.
Stringaris K et al Haematologica. 2014 May; 99(5):836-47. doi: 10.3324/haematol.2013.087536. Epub 2014 Jan. 31. PMID: 24488563; PMCID: PMC4008119.

1. A heterotandem bicyclic peptide complex comprising:
(a) a first peptide ligand which binds to a component present on a cancer cell;
conjugated via a linker to
(b) one or more second peptide ligands which bind to one or more components present on a natural killer (NK) cell;
wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

2. The heterotandem bicyclic peptide complex as defined in claim 1, wherein said reactives group are cysteine residues.

3. The heterotandem bicyclic peptide complex as defined in claim 1, wherein the one or more components present on a natural killer (NK) cell is a natural cytotoxicity receptor present on the NK cell surface, such as NKp30, NKp44 and NKp46, in particular NKp46, such as wherein the one or more second peptide ligands comprise one or more NKp46 binding bicyclic peptide ligands.

4. The heterotandem bicyclic peptide complex as defined in claim 3, wherein the one or more NKp46 binding bicyclic peptide ligands comprise an amino acid sequence which is selected from:
(SEQ ID NO: 5)
CᵢY[Cba]PDYLCᵢᵢ[dA]DEYCᵢᵢᵢ;
(SEQ ID NO: 6)
CᵢYLPDYLCᵢᵢGDEYCᵢᵢᵢ;
(SEQ ID NO: 7)
CᵢDLTTHNCᵢᵢQWGICᵢᵢᵢ;
(SEQ ID NO: 8)
CᵢNLQAPCᵢᵢMQTGKVCᵢᵢᵢ;
(SEQ ID NO: 9)
CᵢNLQNPCᵢᵢMKFPCᵢᵢᵢ;
(SEQ ID NO: 10)
CᵢYLPDYLCᵢᵢ[dK(PYA)]DEYCᵢᵢᵢ;
and
(SEQ ID NO: 11)
CᵢLLHDHCᵢᵢPNTHPKLCᵢᵢᵢ;
wherein Cᵢ, Cᵢᵢ and Cᵢᵢᵢ represent first, second and third cysteine residues, respectively, and wherein Cba represents β-cyclobutylalanine, dA represents D-Alanine, and PYA represents pentynoic acid, or a pharmaceutically acceptable salt thereof, in particular:
wherein the molecular scaffold is TATA and the one or more NKp46 binding bicyclic peptide ligands optionally comprise N-terminal and/or C-terminal modifications and comprises:
Ac-(SEQ ID NO: 5)-[K(PYA)] (herein referred to as BCY17224);
A-(SEQ ID NO: 6)-A-[dK(PYA)] (herein referred to as BCY15452);
A-(SEQ ID NO: 7)-A-[K(PYA)] (herein referred to as BCY15686);
A-(SEQ ID NO: 8)-A-[K(PYA)] (herein referred to as BCY15687);
A-(SEQ ID NO: 9)-A-[K(PYA)] (herein referred to as BCY18004);
A-(SEQ ID NO: 10)-A (herein referred to as BCY17662); and
A-(SEQ ID NO: 11)-A-[K(PYA)] (herein referred to as BCY18005);
wherein PYA represents pentynoic acid or a pharmaceutically acceptable salt thereof.

5. The heterotandem bicyclic peptide complex as defined in claim 1, wherein the one or more components present on a natural killer (NK) cell is an Fc receptor present on the NK cell surface, such as a low-affinity Fc gamma receptor (FcγR) selected from FcγRIIA, FcγRIIB, FcγRIIC, FcγRIIIA, and FcγRIIIB, in particular FcγRIIIA (also known as CD16a), such as wherein the one or more second peptide ligands comprise one or more CD16a binding bicyclic peptide ligands.

6. The heterotandem bicyclic peptide complex as defined in claim 5, wherein the one or more CD16a binding bicyclic peptide ligands comprises an amino acid sequence which is selected from:
(SEQ ID NO: 12)
CᵢVGLEELGPCᵢᵢSDLCᵢᵢᵢ;
(SEQ ID NO: 13)
CᵢRWHFSEPCᵢᵢGAWCᵢᵢᵢ;
and
(SEQ ID NO: 14)
CᵢRWSVEDPCᵢᵢGAWCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TBMT and the one or more CD16a binding bicyclic peptide ligands optionally comprise N-terminal and/or C-terminal modifications and comprises:
A-(SEQ ID NO: 12)-A-[K(PYA)] (herein referred to as BCY13886);
A-(SEQ ID NO: 13)-A-[K(PYA)] (herein referred to as BCY20361); and
A-(SEQ ID NO: 14)-A-[K(PYA)] (herein referred to as BCY13883);
wherein PYA represents pentynoic acid or a pharmaceutically acceptable salt thereof.

7. The heterotandem bicyclic peptide complex as defined in claim 1, wherein the cancer cell is selected from an HT1080, A549, SC-OV-3, PC3, HT1376, NCI-H292, LnCap, MC38, MC38 #13, 4T1-D02, H322, HT29, T47D and RKO tumor cell.

8. The heterotandem bicyclic peptide complex as defined in claim 1, wherein the component present on a cancer cell is EphA2, such as wherein the first peptide comprises an EphA2 binding bicyclic peptide ligand.

9. The heterotandem bicyclic peptide complex as defined in claim 8, wherein the EphA2 binding bicyclic peptide ligand comprises an amino acid sequence which is:
(SEQ ID NO: 2)
Cᵢ[HyP]LVNPLCᵢᵢLHP[dD]W[HArg]Cᵢᵢᵢ;
wherein Cᵢ, Cᵢᵢ and Cᵢᵢᵢ represent first (i), second (ii) and third (iii) reactive groups HyP represents trans-4-hydroxy-L-proline, HArg represents homoarginine, or a pharmaceutically acceptable salt thereof, in particular:
wherein the molecular scaffold is TATA and the EphA2 binding bicyclic peptide ligand optionally comprises N-terminal modifications and comprises:
A-[HArg]-D-(SEQ ID NO: 2) (herein referred to as BCY9594);
wherein HArg represents homoarginine, or a pharmaceutically acceptable salt thereof.

10. The heterotandem bicyclic peptide complex as defined in claim 1, wherein the component present on a cancer cell is PD-L1, such as wherein the first peptide comprises an PD-L1 binding bicyclic peptide ligand.

11. The heterotandem bicyclic peptide complex as defined in claim 10, wherein the PD-L1 binding bicyclic peptide ligand comprises an amino acid sequence which is:
(SEQ ID NO: 3)
CᵢSAGWLTMCᵢᵢQKLHLCᵢᵢᵢ;
wherein Cᵢ, Cᵢᵢ and Cᵢᵢᵢ represent first (i), second (ii) and third (iii) cysteine groups, respectively, or a pharmaceutically acceptable salt thereof, in particular:
wherein the molecular scaffold is TATA and the PD-L1 binding bicyclic peptide ligand optionally comprises N-terminal and/or C-terminal modifications and comprises:
Ac-D-[Harg]-(SEQ ID NO: 3)-PSH (herein referred to as BCY11865);
wherein Harg represents homoarginine, or a pharmaceutically acceptable salt thereof.

12. The heterotandem bicyclic peptide complex as defined in claim 1, wherein the component present on a cancer cell is MT1, such as wherein the first peptide comprises an MT1 binding bicyclic peptide ligand.

13. The heterotandem bicyclic peptide complex as defined in claim 12, wherein the MT1 binding bicyclic peptide ligand comprises an amino acid sequence which is:
(SEQ ID NO: 4)
CᵢV[Harg]ECᵢᵢA[tBuAla]LFP[Harg]TCᵢᵢᵢ;
wherein Cᵢ, Cᵢᵢ and Cᵢᵢᵢ represent first (i), second (ii) and third (iii) cysteine groups, respectively, or a pharmaceutically acceptable salt thereof, in particular:
wherein the molecular scaffold is TATA and the MT1 binding bicyclic peptide ligand optionally comprises N-terminal and/or C-terminal modifications and comprises:
LPP-(SEQ ID NO: 4) (herein referred to as BCY14320);
or a pharmaceutically acceptable salt thereof.

14. The heterotandem bicyclic peptide complex as defined in claim 1, which is any of the complexes listed in Tables A1, A2, A3, B1, C, D and E.

15. The heterotandem bicyclic peptide complex as defined in claim 1, wherein the pharmaceutically acceptable salt is selected from the free acid or the sodium, potassium, calcium, ammonium salt.

16. A pharmaceutical composition which comprises the heterotandem bicyclic peptide complex of claim 1, in combination with one or more pharmaceutically acceptable excipients.

17. A method for preventing, suppressing or treating cancer, in a subject in need thereof, comprising administering to the subject the heterotandem bicyclic peptide complex of claim 1, or a pharmaceutical composition thereof.
